(19) United States
(12) Patent Application Publication
(10) Pub. No.: US 2010/0227313 A1
(43) Pub. Date: Sep. 9, 2010

(54) Methods and Compositions for Determining Altered Susceptibility of HIV-1 to Protease Inhibitor Treatment

(75) Inventors: Neil T. Parkin, Belmont, CA (US); Colombe Chappey, San Francisco, CA (US); Eric Stawiski, San Francisco, CA (US)

Correspondence Address: KILPATRICK STOCKTON LLP, 1001 WEST FOURTH STREET, WINSTON-SALEM, NC, 27101, US

(73) Assignee: LABORATORY CORPORATION OF AMERICA HOLDINGS

(21) Appl. No.: 12/527,861

(22) PCT Filed: Feb. 25, 2008
(86) PCT No.: PCT/US08/02439

§ 371 Date: Mar. 16, 2010

Related U.S. Application Data
(60) Provisional application No. 60/903,103, filed on Feb. 23, 2007.

(51) Int. Cl. C12Q 1/70

(52) U.S. Cl. 435/5

(57) ABSTRACT

This invention relates to methods for determining altered susceptibility of HIV-I viruses to protease inhibitors (PIs) based on the viral genotypes. The methods generally comprise detecting, in a gene encoding protease and/or gag of the HIV-I, the presence of mutations correlated with altered susceptibility to amprenavir and/or darunavir.

### 1. FIELD OF THE INVENTION

This invention relates, in part, to methods for determining the altered susceptibility of a human immunodeficiency virus (“HIV”) to the protease inhibitor darunavir (“DRV”) by detecting the presence of mutations in the genes encoding HIV protease and/or gag that are associated with altered susceptibility to amprenavir (“APV”) and/or DRV.

### 2. BACKGROUND OF THE INVENTION

More than 60 million people have been infected with the human immunodeficiency virus (“HIV”), the causative agent of acquired immune deficiency syndrome (“AIDS”), since the early 1980s. See Lucas, 2002, Lepr Rev. 73(1):64 71. HIV/AIDS is now the leading cause of death in sub Saharan Africa, and is the fourth biggest killer worldwide. At the end of 2001, an estimated 40 million people were living with HIV globally. See Norris, 2002, Radiol Technol. 73(4):339 363.

The goal of antiretroviral therapy drug treatment is to delay disease progression and prolong survival by achieving sustained suppression of viral replication. Current anti-HIV drugs target different stages of the HIV life cycle and a variety of enzymes essential for HIV's replication and/or survival. For example, certain drugs approved for AIDS therapy inhibit HIV replication by interfering with the enzymatic activities of either protease (“PR”) or reverse transcriptase (“RT”). Among the approved drugs are nucleoside RT inhibitors (“NRTIs”) such as AZT, ddI, ddC, d4T, 3TC, abacavir, nucleotide RT inhibitors such as tenofovir, non-nucleoside RT inhibitors (“NNRTIs”) such as nevirapine, efavirenz, delavirdine and protease inhibitors (“PIs”) such as saquinavir, ritonavir, indinavir, nelfinavir, tipranavir, lopinavir, atazanavir, amprenavir and darunavir.

Nonetheless, in the vast majority of subjects none of these antiviral drugs, either alone or in combination, proves effective either to prevent eventual progression of chronic HIV infection to AIDS or to treat acute AIDS. This phenomenon is due, in part, to the high mutation rate of HIV and the rapid emergence of mutant HIV strains that are resistant to antiviral therapeutics upon administration of such drugs to infected individuals.

Many such mutant strains have been characterized in order to correlate presence of the mutations in the strains with resistant or susceptible phenotypes. For example, the L33F mutation in protease is known to correlate with resistance to a number of PIs, including, for example, amprenavir, ritonavir, lopinavir and saquinavir. See, e.g., Kozal et al., 2006, Antivir. Ther. 11(4):457-63. In addition, the I47A mutation is also known to correlate with reduced susceptibility to amprenavir and lopinavir, but with hypersusceptibility to saquinavir. See, e.g., de Mendoza et al., 2006 AIDS 20(7):1071-4. Thus, a given mutation may correlate with resistance to one or more antiviral agent and hypersusceptibility to one or more others.

Though numerous mutations associated with both resistance and susceptibility to particular anti-viral agents have been identified, the effects of these mutations on resistance or susceptibility to other antiviral agents in many cases remains obscure. Thus, an analysis that identifies the effects of mutations associated with resistance to one antiviral agent on resistance or susceptibility to other antiviral agents would be very useful in guiding selection of particular antiviral agents and therapeutic decisions in the treatment of HIV-infected individuals. Further, in view of the clinical relevance of altered PI susceptibility, a more complete understanding of mutations associated with such altered susceptibility is also needed. Thus there remains a need to identify additional mutations associated with altered PI susceptibility and to characterize these mutations.

### 3. SUMMARY OF THE INVENTION

The present invention provides methods for determining whether a human immunodeficiency virus type 1 (“HIV-1”) is likely to have an altered susceptibility to the protease inhibitor darunavir.

Thus, in certain aspects, the invention provides a method for determining whether an HIV-1 is likely to have a reduced susceptibility to darunavir, wherein if the HIV-1 exhibits a reduced susceptibility to amprenavir, the HIV-1 also exhibits a reduced susceptibility to darunavir. In one embodiment, the method comprises determining the susceptibility of the HIV-1 to amprenavir, wherein reduced susceptibility to amprenavir correlates with reduced susceptibility to darunavir, thereby determining whether the HIV-1 is likely to have a reduced susceptibility to darunavir. In certain embodiments, reduced susceptibility to amprenavir is determined by measuring in vitro the phenotypic sensitivity of the HIV-1 to amprenavir. In certain embodiments, reduced susceptibility to amprenavir is determined by detecting, in a gene encoded by the HIV-1, the presence of one or more mutations associated with reduced susceptibility to amprenavir. In certain embodiments, reduced susceptibility to amprenavir is determined by detecting the presence of one or more mutations in at least one of codons 4, 10, 11, 12, 13, 15, 16, 19, 20, 22, 23, 24, 32, 34, 35, 36, 37, 43, 46, 47, 50, 53, 54, 55, 58, 60, 61, 62, 63, 66, 67, 69, 71, 72, 73, 74, 76, 79, 82, 84, 85, 89, 90, 91, 92 and 95 of the protease gene of the HIV-1, wherein the presence of the mutation(s) correlates with reduced susceptibility to amprenavir, thereby determining that the HIV-1 is likely to have reduced sensitivity to darunavir. In certain embodiments, reduced susceptibility to amprenavir is determined by detecting the presence of one or more mutations in at least one of codons 423, 425, 428, 431, 437, 441, 449, 451, 452, 453, 459, 462, 463, 465, 467, 468, 479 and 488 of the gag gene of the HIV-1, wherein the presence of the mutation(s) correlates with reduced susceptibility to amprenavir, thereby determining that the HIV-1 is likely to have reduced sensitivity to darunavir. The presence of the mutations associated with reduced susceptibility to amprenavir can be detected according to any method known to one of skill in the art without limitation. Methods for detecting such mutations are described extensively below.

In other aspects, the invention provides a method for determining whether an HIV-1 is likely to have an increased susceptibility to darunavir, comprising determining the susceptibility of the HIV-1 to amprenavir, wherein increased susceptibility to amprenavir correlates with increased susceptibility to darunavir, thereby determining whether the HIV-1 is likely to have an increased susceptibility to darunavir. In certain embodiments, increased susceptibility to amprenavir is determined by measuring in vitro the phenotypic sensitivity of the HIV-1 to amprenavir. In certain embodiments, increased susceptibility to amprenavir is determined by detecting, in a gene encoded by the HIV-1, the presence of one or more mutations associated with increased susceptibility to amprenavir. In certain embodiments, increased susceptibility to amprenavir is determined by detecting the presence of one or more mutations in at least one of codons 30, 43, 45, 50, 63, 64, 71, 77, 88 and 93 of the protease gene of the HIV-1, wherein the presence of the mutation(s) correlates with increased susceptibility to amprenavir, thereby determining that the HIV-1 is likely to have increased sensitivity to darunavir. In certain embodiments, increased susceptibility to amprenavir is determined by detecting the presence of one or more mutations in at least one of codons 441, 451, 486, 498 and 499 of the gag gene of the HIV-1, wherein the presence of the mutation(s) correlates with increased susceptibility to amprenavir, thereby determining that the HIV-1 is likely to have increased sensitivity to darunavir. The presence of the mutations associated with increased susceptibility to darunavir can be detected according to any method known to one of skill in the art without limitation. Methods for detecting such mutations are described extensively below.

In other aspects, the invention provides a method for determining whether an HIV-1 is likely to have a reduced susceptibility to darunavir, comprising detecting whether an HIV-1 protease mutation is present in at least one of codons 4, 10, 12, 13, 15, 16, 18, 19, 20, 22, 23, 24, 34, 35, 36, 37, 43, 46, 48, 53, 55, 57, 58, 60, 61, 62, 63, 66, 67, 69, 71, 72, 74, 79, 82, 83, 85, 90, 91, 92, and 95 of the HIV-1, wherein the presence of the mutation(s) correlates with reduced susceptibility to treatment with darunavir, thereby determining whether the HIV-1 is likely to have a reduced susceptibility to darunavir. In certain embodiments, the methods comprise detecting whether an HIV-1 protease mutation is present in at least one of codons 4, 10, 12, 13, 15, 16, 18, 19, 20, 22, 23, 24, 34, 35, 36, 37, 43, 46, 48, 53, 55, 57, 58, 60, 61, 62, 63, 66, 67, 69, 71, 72, 74, 79, 82, 83, 85, 90, 91, 92, and 95 in combination with a mutation in at least one of codons 11, 32, 33, 47, 50, 54, 73, 76, 84 and 89, wherein the presence of the mutations correlate with reduced susceptibility to treatment with darunavir, thereby determining whether the HIV-1 is likely to have a reduced susceptibility to darunavir. In certain embodiments, the methods comprise detecting whether an HIV-1 gag mutation is present in at least one of codons 423, 425, 428, 431, 435, 437, 441, 449, 451, 452, 453, 459, 462, 463, 465, 467, 468, 469, 479, 488 and 497 of the HIV-1, wherein the presence of the mutation(s) correlates with reduced susceptibility to treatment with darunavir, thereby determining whether the HIV-1 is likely to have a reduced susceptibility to darunavir. The presence of the mutations associated with reduced susceptibility to darunavir can be detected according to any method known to one of skill in the art without limitation. Methods for detecting such mutations are described extensively below.

In other aspects, the invention provides a method for determining whether an HIV-1 is likely to have an increased susceptibility to darunavir, wherein if the HIV-1 exhibits an increased susceptibility to amprenavir, the HIV-1 also exhibits an increased susceptibility to darunavir. In one embodiment, the method comprises detecting whether an HIV-1 protease mutation is present in at least one of codons 20, 30, 36, 41, 43, 45, 50, 63, 64, 65, 70, 71, 74, 75, 77, 82, 88 and 93 of the HIV-1, wherein the presence of the mutation(s) correlates with increased susceptibility to treatment with darunavir, thereby determining whether the HIV-1 is likely to have an increased susceptibility to darunavir. In certain embodiments, the methods comprise detecting whether an HIV-1 protease mutation is present in at least one of codons 20, 30, 36, 41, 43, 45, 50, 63, 64, 65, 70, 71, 74, 75, 77, 82, 88 and 93 in combination with a mutation in at least one of codons 30, 50 or 88, wherein the presence of the mutations correlate with increased susceptibility to treatment with darunavir, thereby determining whether the HIV-1 is likely to have an increased susceptibility to darunavir. In certain embodiments, the methods comprise detecting whether an HIV-1 gag mutation is present in at least one of codons 437, 439, 441, 442, 451, 475, 480, 482, 483, 486, 498 and 499 of the HIV-1, wherein the presence of the mutation(s) correlates with increased susceptibility to treatment with darunavir, thereby determining whether the HIV-1 is likely to have an increased susceptibility to darunavir. The presence of the mutations associated with increased susceptibility to amprenavir can be detected according to any method known to one of skill in the art without limitation. Methods for detecting such mutations are described extensively below.

In other aspects, the invention provides a method for determining whether an HIV-1 is likely to have a reduced susceptibility to amprenavir, comprising detecting whether an HIV-1 protease mutation is present in at least one of codons 4, 10, 12, 13, 15, 16, 18, 19, 20, 22, 23, 24, 34, 35, 36, 37, 43, 46, 48, 53, 55, 57, 58, 60, 61, 62, 63, 66, 67, 69, 71, 72, 74, 79, 82, 83, 85, 90, 91, 92, and 95 of the HIV-1, wherein the presence of the mutation(s) correlates with reduced susceptibility to treatment with darunavir, thereby determining whether the HIV-1 is likely to have a reduced susceptibility to amprenavir. In certain embodiments, the methods comprise detecting whether an HIV-1 protease mutation is present in at least one of codons 4, 10, 12, 13, 15, 16, 18, 19, 20, 22, 23, 24, 34, 35, 36, 37, 43, 46, 48, 53, 55, 57, 58, 60, 61, 62, 63, 66, 67, 69, 71, 72, 74, 79, 82, 83, 85, 90, 91, 92, and 95 in combination with a mutation in at least one of codons 11, 32, 33, 47, 50, 54, 73, 76, 84 and 89, wherein the presence of the mutations correlate with reduced susceptibility to treatment with darunavir, thereby determining whether the HIV-1 is likely to have a reduced susceptibility to amprenavir. In certain embodiments, the methods comprise detecting whether an HIV-1 gag mutation is present in at least one of codons 423, 425, 428, 431, 435, 437, 441, 449, 451, 452, 453, 459, 462, 463, 465, 467, 468, 469, 479, 488 and 497 of the HIV-1, wherein the presence of the mutation(s) correlates with reduced susceptibility to treatment with darunavir, thereby determining whether the HIV-1 is likely to have a reduced susceptibility to amprenavir. The presence of the mutations associated with reduced susceptibility to darunavir can be detected according to any method known to one of skill in the art without limitation. Methods for detecting such mutations are described extensively below.

In still other aspects, the invention provides a method for determining whether an HIV-1 is likely to have an increased susceptibility to amprenavir, wherein if the HIV-1 exhibits an increased susceptibility to darunavir, the HIV-1 also exhibits an increased susceptibility to amprenavir. In one embodiment, the method comprises detecting whether an HIV-1 protease mutation is present in at least one of codons 20, 30, 36, 41, 43, 45, 50, 63, 64, 65, 70, 71, 74, 75, 77, 82, 88 and 93 of the HIV-1, wherein the presence of the mutation(s) correlates with increased susceptibility to treatment with darunavir, thereby determining whether the HIV-1 is likely to have an increased susceptibility to amprenavir. In certain embodiments, the methods comprise detecting whether an HIV-1 protease mutation is present in at least one of codons 20, 30, 36, 41, 43, 45, 50, 63, 64, 65, 70, 71, 74, 75, 77, 82, 88 and 93 in combination with a mutation in at least one of codons 30, 50 or 88, wherein the presence of the mutations correlate with increased susceptibility to treatment with darunavir, thereby determining whether the HIV-1 is likely to have an increased susceptibility to amprenavir. In certain embodiments, the methods comprise detecting whether an HIV-1 gag mutation is present in at least one of codons 437, 439, 441, 442, 451, 475, 480, 482, 483, 486, 498 and 499 of the HIV-1, wherein the presence of the mutation(s) correlates with increased susceptibility to treatment with darunavir, thereby determining whether the HIV-1 is likely to have an increased susceptibility to amprenavir. The presence of the mutations associated with increased susceptibility to darunavir can be detected according to any method known to one of skill in the art without limitation. Methods for detecting such mutations are described extensively below.

### 4. BRIEF DESCRIPTION OF THE FIGURES

FIG. 1A presents the distribution of fold change in IC₅₀ observed in the presence of amprenavir (APV) plotted against the fold change in IC₅₀ observed in the presence of darunavir (DRV) for HIV-1 isolated from 2862 clinical samples comprising at least one major PI resistance-associated mutation at positions L23, L24, D30, V32, M46, I47, G48, I50, I54, V82 (except V82I), I84, N88, and L90, with no mixtures at V11I, V32I, L33F, I47V, I50V, I54L or M, G73S, L76V, I84V and L89Vin the HIV-1 protease.

FIG. 1B presents the distribution of fold change in IC₅₀ observed in the presence of lopinavir (LPV) plotted against the fold change in IC₅₀ observed in the presence of darunavir (DRV) for HIV-1 isolated from 2862 clinical samples comprising at least one major PI resistance-associated mutation at positions L23, L24, D30, V32, M46, I47, G48, I50, I54, V82 (except V821), 184, N88, and L90, with no mixtures at V11I, V32I, L33F, I47V, I50V, I54L or M, G73S, L76V, 184V and L89V in the HIV-1 protease.

FIG. 1C presents the distribution of fold change in IC₅₀ observed in the presence of atazanavir (ATV) plotted against the fold change in IC₅₀ observed in the presence of darunavir (DRV) for HIV-1 isolated from 2862 clinical samples comprising at least one major PI resistance-associated mutation at positions L23, L24, D30, V32, M46, I47, G48, I50, I54, V82 (except V82I), 184, N88, and L90, with no mixtures at V11I, V32I, L33F, 147V, I50V, I54L or M, G73S, L76V, 184V and L89V in the HIV-1 protease.

FIG. 1D presents the distribution of fold change in IC₅₀ observed in the presence of tipranavir (TPV) plotted against the fold change in IC₅₀ observed in the presence of darunavir (DRV) for HIV-1 isolated from 2862 clinical samples comprising at least one major PI resistance-associated mutation at positions L23, L24, D30, V32, M46, I47, G48, I50, I54, V82 (except V82I), 184, N88, and L90, with no mixtures at V11I, V32I, L33F, 147V, I50V, I54L or M, G73S, L76V, 184V and L89V in the HIV-1 protease.

### 5. DETAILED DESCRIPTION OF THE INVENTION

The present invention provides methods for determining that an HIV-1 has reduced or increased susceptibility to antiviral therapy with DRV. The methods generally comprise detecting the presence of mutations in the HIV-1 gene encoding protease or gag that significantly correlate with reduced or increased susceptibility to APV or DRV.

### 5.1 Abbreviations

“NRTI” is an abbreviation for nucleoside reverse transcriptase inhibitor.

“NNRTI” is an abbreviation for non nucleoside reverse transcriptase inhibitor.

“PI” is an abbreviation for protease inhibitor.

“PR” is an abbreviation for protease.

“RT” is an abbreviation for reverse transcriptase.

“PCR” is an abbreviation for “polymerase chain reaction.”

“HBV” is an abbreviation for hepatitis B virus.

“HCV” is an abbreviation for hepatitis C virus.

“HIV” is an abbreviation for human immunodeficiency virus.

“DRV” is an abbreviation for the PI darunavir.

“APV” is an abbreviation for the PI amprenavir.

“LPV” is an abbreviation for the PI lopinavir.

“ATV” is an abbreviation for the PI atazanavir.

“TPV” is an abbreviation for the PI tipranavir.

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless noted otherwise, when polypeptide sequences are presented as a series of one-letter and/or three-letter abbreviations, the sequences are presented in the N->C direction, in accordance with common practice.

Individual amino acids in a sequence are represented herein as AN, wherein A is the standard one letter symbol for the amino acid in the sequence, and N is the position in the sequence. Mutations are represented herein as A1NA2, wherein A1 is the standard one letter symbol for the amino acid in the reference protein sequence, A2 is the standard one letter symbol for the amino acid in the mutated protein sequence, and N is the position in the amino acid sequence. For example, a G25M mutation represents a change from glycine to methionine at amino acid position 25. Mutations may also be represented herein as NA2, wherein N is the position in the amino acid sequence and A2 is the standard one letter symbol for the amino acid in the mutated protein sequence (e.g., 25M, for a change from the wild-type amino acid to methionine at amino acid position 25). Additionally, mutations may also be represented herein as A 1NX, wherein A1 is the standard one letter symbol for the amino acid in the reference protein sequence, N is the position in the amino acid sequence, and X indicates that the mutated amino acid can be any amino acid (e.g., G25X represents a change from glycine to any amino acid at amino acid position 25). This notation is typically used when the amino acid in the mutated protein sequence is either not known or, if the amino acid in the mutated protein sequence could be any amino acid, except that found in the reference protein sequence. The amino acid positions are numbered based on the full length sequence of the protein from which the region encompassing the mutation is derived. Representations of nucleotides and point mutations in DNA sequences are analogous.

The abbreviations used throughout the specification to refer to nucleic acids comprising specific nucleobase sequences are the conventional one letter abbreviations. Thus, when included in a nucleic acid, the naturally occurring encoding nucleobases are abbreviated as follows: adenine (A), guanine (G), cytosine (C), thymine (T) and uracil (U). Unless specified otherwise, single-stranded nucleic acid sequences that are represented as a series of one letter abbreviations, and the top strand of double-stranded sequences, are presented in the 5′->3′ direction.

### 5.2 DEFINITIONS

As used herein, the following terms shall have the following meanings:

A “phenotypic assay” is a test that measures a phenotype of a particular virus, such as, for example, HIV, or a population of viruses, such as, for example, the population of HIV infecting a subject. The phenotypes that can be measured include, but are not limited to, the resistance or susceptibility of a virus, or of a population of viruses, to a specific anti-viral agent or that measures the replication capacity of a virus.

“Susceptibility” refers to a virus' response to a particular drug. A virus that is less susceptible or has decreased susceptibility to a drug is less sensitive or more resistant to the drug. A virus that has increased or enhanced or greater susceptibility to a drug has an increased sensitivity or decreased resistance to the drug.

Phenotypic drug susceptibility is measured as the concentration of drug required to inhibit virus replication by 50% (IC₅₀). As used herein, a “fold change” or “FC” is the ratio of a viral variant IC₅₀ divided by the IC₅₀ of a reference HIV. An FC of 1.0 indicates that the viral variant exhibits the same degree of drug susceptibility as the reference virus.

A “fold change” is a numeric comparison of the drug susceptibility of a patient virus and a drug-sensitive reference virus. It is the ratio of the Patient IC₅₀ to the drug-sensitive reference IC₅₀, i.e., Patient IC₅₀/Reference IC₅₀=Fold Change (“FC”). A fold change of 1.0 indicates that the patient virus exhibits the same degree of drug susceptibility as the drug-sensitive reference virus. A fold change less than 1 indicates the patient virus is more sensitive than the drug-sensitive reference virus. A fold change greater than 1 indicates the patient virus is less susceptible than the drug-sensitive reference virus. A fold change equal to or greater than the clinical cutoff value means the patient virus has a lower probability of response to that drug. A fold change less than the clinical cutoff value means the patient virus is sensitive to that drug.

An “odds ratio” (OR) is a numeric comparison of the percentage of samples which demonstrate altered susceptibility to a drug and harbor a particular mutation versus the percentage of samples which do not display altered susceptibility to the drug and harbor the same mutation.

A “genotypic assay” is an assay that determines a genotype of an organism, a part of an organism, a population of organisms, a gene, a part of a gene, or a population of genes. Typically, a genotypic assay involves determination of the nucleic acid sequence of the relevant gene or genes. Such assays are frequently performed in HIV to establish, for example, whether certain mutations are associated with drug resistance or hypersusceptibility or altered replication capacity are present.

As used herein, “genotypic data” are data about the genotype of, for example, a virus. Examples of genotypic data include, but are not limited to, the nucleotide or amino acid sequence of a virus, a population of viruses, a part of a virus, a viral gene, a part of a viral gene, or the identity of one or more nucleotides or amino acid residues in a viral nucleic acid or protein.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. Exemplary levels of sequence identity include, but are not limited to, 60, 70, 80, 85, 90, 95, 98% or more sequence identity to a given sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. Exemplary levels of sequence homology include, but are not limited to, 60, 70, 80, 85, 90, 95, 98% or more sequence homology to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See id.

A preferred alignment of selected sequences in order to determine “% identity” between two or more sequences, is performed using for example, the CLUSTAL-X program, operated with default parameters, including an open gap penalty of 10.0, an extended gap penalty of 0.1, and a BLOSUM 30 similarity matrix.

“Polar Amino Acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

“Nonpolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Ala (A), Gly (G), Ile (I), Leu (L), Met (M) and Val (V).

“Hydrophilic Amino Acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Arg (R), Asn (N), Asp (D), Glu (E), Gln (O), H is (H), Lys (K), Ser (S) and Thr (T).

“Hydrophobic Amino Acid” refers to an amino acid exhibiting a hydrophobicity of greater than zero according to the normalized consensus hydrophobicity scale of Eisenberg et al., 1984, J. Mol. Biol. 179:125-142. Genetically encoded hydrophobic amino acids include Ala (A), Gly (G), Ile (I), Leu (L), Met (M), Phe (F), Pro (P), Trp (W), Tyr (Y) and Val (V).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Asp (D) and Glu (E).

“Basic Amino Acid” refers to a hydrophilic amino acid having a side chain pK value of greater than 7. Basic amino acids typically have positively charged side chains at physiological pH due to association with a hydrogen ion. Genetically encoded basic amino acids include Arg (R), H is (H) and Lys (K).

A “mutation” is a change in an amino acid sequence or in a corresponding nucleic acid sequence relative to a reference nucleic acid or polypeptide. For embodiments of the invention comprising HIV polypeptide or nucleic acid sequences, e.g., protease, reverse transcriptase, gag pol or env sequences, the reference nucleic acid encoding such HIV sequences is the HIV coding sequencepresent in NL4-3 HIV (GenBank Accession No. AF324493). Likewise, the reference HIV polypeptide is that encoded by the NL4-3 HIV sequence. Of course, one skilled in the art will recognize that the position of the amino acid mutations in test viruses may vary based on insertions, deletions and the like. One skilled in the art may routinely identify positions corresponding to particular amino acid or nucleic acid positions of the reference virus using techniques routine to the art, e.g. the alignment algorithms described herein. Although the amino acid sequence of a peptide can be determined directly by, for example, Edman degradation or mass spectroscopy, more typically, the amino sequence of a peptide is inferred from the nucleotide sequence of a nucleic acid that encodes the peptide. Any method for determining the sequence of a nucleic acid known in the art can be used, for example, Maxam-Gilbert sequencing (Maxam et al., 1980, Methods in Enzymology 65:499), dideoxy sequencing (Sanger et al., 1977, Proc. Natl. Acad. Sci. USA 74:5463) or hybridization-based approaches (see e.g., Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY).

A “mutant” is a virus, gene or protein having a sequence that has one or more changes relative to a reference virus, gene or protein.

The terms “peptide,” “polypeptide” and “protein” are used interchangeably throughout.

The term “wild-type” refers to a viral genotype that does not comprise a mutation known to be associated with drug resistance.

The terms “polynucleotide,” “oligonucleotide” and “nucleic acid” are used interchangeably throughout.

### 5.3 Methods of Determining Reduced Susceptibility to DRV

In certain aspects, the present invention provides methods for determining whether an HIV-1 has reduced susceptibility to DRV. In general, the methods comprise detecting whether mutations significantly correlated with reduced susceptibility to APV or DRV are present in the gene encoding protease or gag of the HIV-1, as demonstrated by the examples below.

Therefore, in certain embodiments, the invention provides a method for determining whether an HIV-1 is likely to have a reduced susceptibility to darunavir, wherein if the HIV-1 exhibits a reduced susceptibility to amprenavir, the HIV-1 also exhibits a reduced susceptibility to darunavir. In one embodiment, the method comprises determining the susceptibility of the HIV-1 to amprenavir, wherein reduced susceptibility to amprenavir correlates with reduced susceptibility to darunavir, thereby determining whether the HIV-1 is likely to have a reduced susceptibility to darunavir. In certain embodiments, reduced susceptibility to amprenavir is determined by measuring in vitro the phenotypic sensitivity of the HIV-1 to amprenavir. In certain embodiments, reduced susceptibility to amprenavir is determined by detecting, in a gene encoded by the HIV-1, the presence of one or more mutations associated with reduced susceptibility to amprenavir.

### 5.3.1 Mutations in HIV-1 Protease Which Correlate with Reduced Susceptibility to APV

In certain embodiments, reduced susceptibility to amprenavir is determined by detecting the presence of one or more mutations in at least one of codons 4, 10, 11, 12, 13, 15, 16, 19, 20, 22, 23, 24, 32, 34, 35, 36, 37, 43, 46, 47, 50, 53, 54, 55, 58, 60, 61, 62, 63, 66, 67, 69, 71, 72, 73, 74, 76, 79, 82, 84, 85, 89, 90, 91, 92 and 95 of the protease gene of the HIV-1, wherein the presence of the mutation(s) correlates with reduced susceptibility to amprenavir, thereby determining that the HIV-1 is likely to have reduced sensitivity to darunavir.

In certain embodiments, a mutation at codon 4 is detected. In certain embodiments, the mutation at codon 4 encodes alanine (A), proline (P) or serine (S). In certain embodiments, a mutation at codon 10 is detected. In certain embodiments, the mutation at codon 10 encodes phenylalanine (F), valine (V) or isoleucine (I). In certain embodiments, a mutation at codon 11 is detected. In certain embodiments, the mutation at codon 11 leucine (L) or isoleucine (I). In certain embodiments, a mutation at codon 12 is detected. In certain embodiments, the mutation at codon 12 encodes alanine proline (P). In certain embodiments, a mutation at codon 13 is detected. In certain embodiments, the mutation at codon 13 encodes valine (V). In certain embodiments, a mutation at codon 15 is detected. In certain embodiments, the mutation at codon 15 encodes valine (V). In certain embodiments, a mutation at codon 16 is detected. In certain embodiments, the mutation at codon 16 encodes alanine (A). In certain embodiments, a mutation at codon 19 is detected. In certain embodiments, the mutation at codon 19 encodes proline (P). In certain embodiments, a mutation at codon 20 is detected. In certain embodiments, the mutation at codon 20 encodes valine (V), arginine (R) or threonine (T). In certain embodiments, a mutation at codon 22 is detected. In certain embodiments, the mutation at codon 22 encodes valine (V). In certain embodiments, a mutation at codon 23 is detected. In certain embodiments, the mutation at codon 23 encodes isoleucine (I). In certain embodiments, a mutation at codon 24 is detected. In certain embodiments, the mutation at codon 24 encodes phenylalanine (F). In certain embodiments, a mutation at codon 32 is detected. In certain embodiments, the mutation at codon 32 encodes isoleucine (I). In certain embodiments, a mutation at codon 33 is detected. In certain embodiments, the mutation at codon 33 encodes phenylalanine (F). In certain embodiments, a mutation at codon 34 is detected. In certain embodiments, the mutation at codon 34 encodes glutamine (Q). In certain embodiments, a mutation at codon 35 is detected. In certain embodiments, the mutation at codon 35 encodes asparagine (N) or aspartic acid (D). In certain embodiments, a mutation at codon 36 is detected. In certain embodiments, the mutation at codon 36 encodes leucine (L) or isoleucine (I). In certain embodiments, a mutation at codon 37 is detected. In certain embodiments, the mutation at codon 37 encodes aspartic acid (D). In certain embodiments, a mutation at codon 43 is detected. In certain embodiments, the mutation at codon 43 encodes threonine (T). In certain embodiments, a mutation at codon 46 is detected. In certain embodiments, the mutation at codon 46 encodes isoleucine (I) or leucine (L). In certain embodiments, a mutation at codon 47 is detected. In certain embodiments, the mutation at codon 47 encodes valine (V). In certain embodiments, a mutation at codon 50 is detected. In certain embodiments, the mutation at codon 50 encodes valine (V). In certain embodiments, a mutation at codon 53 is detected. In certain embodiments, the mutation at codon 53 encodes tyrosine (Y) or leucine (L). In certain embodiments, a mutation at codon 54 is detected. In certain embodiments, the mutation at codon 54 encodes methionine (M), leucine (L), serine (S), threonine (T), alanine (A) or valine (V). In certain embodiments, a mutation at codon 55 is detected. In certain embodiments, the mutation at codon 55 encodes asparagines (N) or arginine (R). In certain embodiments, a mutation at codon 58 is detected. In certain embodiments, the mutation at codon 58 encodes glutamic acid (E). In certain embodiments, a mutation at codon 60 is detected. In certain embodiments, the mutation at codon 60 encodes glutamic acid (E). In certain embodiments, a mutation at codon 61 is detected. In certain embodiments, the mutation at codon 61 encodes asparagine (N). In certain embodiments, a mutation at codon 62 is detected. In certain embodiments, the mutation at codon 62 encodes valine (V). In certain embodiments, a mutation at codon 63 is detected. In certain embodiments, the mutation at codon 63 encodes proline (P). In certain embodiments, a mutation at codon 66 is detected. In certain embodiments, the mutation at codon 66 encodes valine (V) or phenylalanine (F). In certain embodiments, a mutation at codon 67 is detected. In certain embodiments, the mutation at codon 67 encodes phenylalanine (F). In certain embodiments, a mutation at codon 69 is detected. In certain embodiments, the mutation at codon 69 encodes arginine (R). In certain embodiments, a mutation at codon 71 is detected. In certain embodiments, the mutation at codon 71 encodes leucine (L), isoleucine (I) or valine (V). In certain embodiments, a mutation at codon 72 is detected. In certain embodiments, the mutation at codon 72 encodes leucine (L) or valine (V). In certain embodiments, a mutation at codon 73 is detected. In certain embodiments, the mutation at codon 73 encodes alanine (A), threonine (T), cysteine (C) or serine (S). In certain embodiments, a mutation at codon 74 is detected. In certain embodiments, the mutation at codon 74 encodes proline (P). In certain embodiments, a mutation at codon 76 is detected. In certain embodiments, the mutation at codon 76 encodes valine (V). In certain embodiments, a mutation at codon 79 is detected. In certain embodiments, the mutation at codon 79 encodes alanine (A) or serine (S). In certain embodiments, a mutation at codon 82 is detected. In certain embodiments, the mutation at codon 82 encodes phenylalanine (F), leucine (L), cysteine (C), isoleucine (I), threonine (T) or alanine (A). In certain embodiments, a mutation at codon 84 is detected. In certain embodiments, the mutation at codon 84 encodes valine (V). In certain embodiments, a mutation at codon 85 is detected. In certain embodiments, the mutation at codon 85 encodes valine (V). In certain embodiments, a mutation at codon 89 is detected. In certain embodiments, the mutation at codon 89 encodes valine (V), isoleucine (I) or methionine (M). In certain embodiments, a mutation at codon 90 is detected. In certain embodiments, the mutation at codon 90 encodes methionine (M). In certain embodiments, a mutation at codon 91 is detected. In certain embodiments, the mutation at codon 91 encodes serine (S). In certain embodiments, a mutation at codon 92 is detected. In certain embodiments, the mutation at codon 92 encodes lysine (K). In certain embodiments, a mutation at codon 95 is detected. In certain embodiments, the mutation at codon 95 encodes valine (V).

### 5.3.2 Mutations in HIV-1 Gag which Correlate with Reduced Susceptibility to APV

In certain embodiments, reduced susceptibility to amprenavir is determined by detecting the presence of one or more mutations in at least one of codons 423, 425, 428, 431, 437, 441, 449, 451, 452, 453, 459, 462, 463, 465, 467, 468, 479 and 488 of the gag gene of the HIV-1, wherein the presence of the mutation(s) correlates with reduced susceptibility to amprenavir, thereby determining that the HIV-1 is likely to have reduced sensitivity to darunavir.

In certain embodiments, a mutation at codon 423 is detected. In certain embodiments, the mutation at codon 423 encodes isoleucine (I). In certain embodiments, a mutation at codon 425 is detected. In certain embodiments, the mutation at codon 425 encodes glutamic acid (E). In certain embodiments, a mutation at codon 428 is detected. In certain embodiments, the mutation at codon 428 encodes aspartic acid (D). In certain embodiments, a mutation at codon 431 is detected. In certain embodiments, the mutation at codon 431 encodes isoleucine (I) or valine (V). In certain embodiments, a mutation at codon 437 is detected. In certain embodiments, the mutation at codon 437 encodes valine (V). In certain embodiments, a mutation at codon 441 is detected. In certain embodiments, the mutation at codon 441 encodes glutamine (Q). In certain embodiments, a mutation at codon 449 is detected. In certain embodiments, the mutation at codon 449 encodes valine (V) or phenylalanine (F). In certain embodiments, a mutation at codon 451 is detected. In certain embodiments, the mutation at codon 451 encodes threonine (T). In certain embodiments, a mutation at codon 452 is detected. In certain embodiments, the mutation at codon 452 encodes serine (S) or lysine (K). In certain embodiments, a mutation at codon 453 is detected. In certain embodiments, the mutation at codon 453 encodes valine (V), isoleucine (I) or leucine (L). In certain embodiments, a mutation at codon 459 is detected. In certain embodiments, the mutation at codon 459 encodes threonine (T). In certain embodiments, a mutation at codon 462 is detected. In certain embodiments, the mutation at codon 462 encodes asparagine (N). In certain embodiments, a mutation at codon 463 is detected. In certain embodiments, the mutation at codon 463 encodes serine (S), valine (V) or leucine (L). In certain embodiments, a mutation at codon 465 is detected. In certain embodiments, the mutation at codon 465 encodes serine (S) or leucine (L). In certain embodiments, a mutation at codon 467 is detected. In certain embodiments, the mutation at codon 467 encodes lysine (K). In certain embodiments, a mutation at codon 468 is detected. In certain embodiments, the mutation at codon 468 encodes lysine (K). In certain embodiments, a mutation at codon 479 is detected. In certain embodiments, the mutation at codon 479 encodes threonine (T). In certain embodiments, a mutation at codon 488 is detected. In certain embodiments, the mutation at codon 488 encodes alanine (A).

### 5.3.3 Mutations in HIV-1 Protease which Correlate with Reduced Susceptibility to DRV

In another aspect, the invention provides a method for determining whether an HIV-1 is likely to have a reduced susceptibility to darunavir, comprising detecting whether an HIV-1 protease mutation is present in at least one of codons 4, 10, 12, 13, 15, 16, 18, 19, 20, 22, 23, 24, 34, 35, 36, 37, 43, 46, 48, 53, 55, 57, 58, 60, 61, 62, 63, 66, 67, 69, 71, 72, 74, 79, 82, 83, 85, 90, 91, 92, and 95 of the HIV-1, wherein the presence of the mutation(s) correlates with reduced susceptibility to treatment with darunavir, thereby determining whether the HIV-1 is likely to have a reduced susceptibility to darunavir.

In certain embodiments, a mutation at codon 4 is detected. In certain embodiments, the mutation at codon 4 encodes alanine (A) or proline (P). In certain embodiments, a mutation at codon 16 is detected. In certain embodiments, the mutation at codon 16 encodes alanine (A). In certain embodiments, a mutation at codon 19 is detected. In certain embodiments, the mutation at codon 19 encodes proline (P). In certain embodiments, a mutation at codon 20 is detected. In certain embodiments, the mutation at codon 20 encodes valine (V). In certain embodiments, a mutation at codon 22 is detected. In certain embodiments, the mutation at codon 22 encodes valine (V). In certain embodiments, a mutation at codon 24 is detected. In certain embodiments, the mutation at codon 24 encodes phenylalanine (F). In certain embodiments, a mutation at codon 34 is detected. In certain embodiments, the mutation at codon 34 encodes glutamine (Q). In certain embodiments, a mutation at codon 43 is detected. In certain embodiments, the mutation at codon 43 encodes threonine (T). In certain embodiments, a mutation at codon 53 is detected. In certain embodiments, the mutation at codon 53 encodes tyrosine (Y). In certain embodiments, a mutation at codon 55 is detected. In certain embodiments, the mutation at codon 55 encodes asparagine (N). In certain embodiments, a mutation at codon 66 is detected. In certain embodiments, the mutation at codon 66 encodes valine (V) or phenylalanine (F). In certain embodiments, a mutation at codon 67 is detected. In certain embodiments, the mutation at codon 67 encodes phenylalanine (F). In certain embodiments, a mutation at codon 71 is detected. In certain embodiments, the mutation at codon 71 encodes leucine (L). In certain embodiments, a mutation at codon 72 is detected. In certain embodiments, the mutation at codon 72 encodes leucine (L). In certain embodiments, a mutation at codon 74 is detected. In certain embodiments, the mutation at codon 74 encodes proline (P). In certain embodiments, a mutation at codon 79 is detected. In certain embodiments, the mutation at codon 79 encodes alanine (A) or serine (S). In certain embodiments, a mutation at codon 82 is detected. In certain embodiments, the mutation at codon 82 encodes phenylalanine (F), leucine (L) or cysteine (C). In certain embodiments, a mutation at codon 91 is detected. In certain embodiments, the mutation at codon 91 encodes serine (S). In certain embodiments, a mutation at codon 95 is detected. In certain embodiments, the mutation at codon 95 encodes phenylalanine (95).

In certain embodiments, the methods further comprise detecting whether a mutation in at least one of codons 4, 10, 12, 13, 15, 16, 18, 19, 20, 22, 23, 24, 34, 35, 36, 37, 43, 46, 48, 53, 55, 57, 58, 60, 61, 62, 63, 66, 67, 69, 71, 72, 74, 79, 82, 83, 85, 90, 91, 92, and 95 is present in combination with at least one of codons 11, 32, 33, 47, 50, 54, 73, 76, 84 and 89 of the HIV-1. In certain embodiments, a combination of mutations is detected comprising a mutation at codon 11. In certain embodiments, the mutation at codon 11 encodes isoleucine (I). In certain embodiments, a combination of mutations is detected comprising a mutation at codon 32. In certain embodiments, the mutation at codon 32 encodes isoleucine (I). In certain embodiments, a combination of mutations is detected comprising a mutation at codon 33. In certain embodiments, the mutation at codon 33 encodes phenylalanine (F). In certain embodiments, a combination of mutations is detected comprising a mutation at codon 47. In certain embodiments, the mutation at codon 47 encodes valine (V). In certain embodiments, a combination of mutations is detected comprising a mutation at codon 50. In certain embodiments, the mutation at codon 50 encodes valine (V). In certain embodiments, a combination of mutations is detected comprising a mutation at codon 54. In certain embodiments, the mutation at codon 54 encodes leucine (L), methionine (M), serine (S) or threonine (T). In certain embodiments, a combination of mutations is detected comprising a mutation at codon 73. In certain embodiments, the mutation at codon 73 encodes alanine (A), serine (S) or threonine (T). In certain embodiments, a combination of mutations is detected comprising a mutation at codon 76. In certain embodiments, the mutation at codon 76 encodes valine (V). In certain embodiments, a combination of mutations is detected comprising a mutation at codon 84. In certain embodiments, the mutation at codon 84 encodes valine (V). In certain embodiments, a combination of mutations is detected comprising a mutation at codon 89. In certain embodiments, the mutation at codon 89 encodes valine (V).

In certain embodiments, the HIV-1 is determined to have a reduced susceptibility to darunavir by detecting an HIV-1 protease, wherein the mutation is significantly associated with reduced susceptibility to DRV, as indicated in Table 1. In certain embodiments, the significance of the association of the mutation with reduced DRV susceptibility is indicated by an odds ratio greater than 1. In certain embodiments, the mutation has an odds ratio greater than 2. In certain embodiments, the mutation has an odds ratio greater than 3. In certain embodiments, the significance of the association of the mutation with reduced DRV susceptibility is indicated by a p-value calculated in Fisher's Exact test (including the Benjamini correction for multiple comparisons) of less than 0.05. In certain embodiments, the significance of the association of the mutation with reduced DRV susceptibility is indicated by a p-value calculated in Fisher's Exact test of less than 0.01.

### 5.3.4 Mutations in HIV-1 Gag which Correlate with Reduced Susceptibility to DRV

In another aspect, the invention provides a method for determining whether an HIV-1 is likely to have a reduced susceptibility to darunavir, comprising detecting whether an HIV-1 gag mutation is present in at least one of codons 423, 425, 428, 431, 435, 437, 441, 449, 451, 452, 453, 459, 462, 463, 465, 467, 468, 469, 479, 488 and 497 of the HIV-1, wherein the presence of the mutation(s) correlates with reduced susceptibility to treatment with darunavir, thereby determining whether the HIV-1 is likely to have a reduced susceptibility to darunavir.

In certain embodiments, a mutation at codon 431 is detected. In certain embodiments, the mutation at codon 431 encodes isoleucine (I). In certain embodiments, a mutation at codon 449 is detected. In certain embodiments, the mutation at codon 449 encodes valine (V). In certain embodiments, a mutation at codon 452 is detected. In certain embodiments, the mutation at codon 452 encodes serine (S). In certain embodiments, a mutation at codon 453 is detected. In certain embodiments, the mutation at codon 453 encodes valine (V) or isoleucine (I). In certain embodiments, a mutation at codon 463 is detected. In certain embodiments, the mutation at codon 463 glutamic acid (E) or serine (S).

In certain embodiments, the HIV-1 is determined to have a reduced susceptibility to darunavir by detecting an HIV-1 gag mutation, wherein the mutation is significantly associated with reduced susceptibility to DRV, as indicated in Table 1. In certain embodiments, the significance of the association of the mutation with reduced DRV susceptibility is indicated by an odds ratio greater than 1. In certain embodiments, the mutation has an odds ratio greater than 2. In certain embodiments, the mutation has an odds ratio greater than 3. In certain embodiments, the significance of the association of the mutation with reduced DRV susceptibility is indicated by a p-value calculated in Fisher's Exact test (including the Benjamini correction for multiple comparisons) of less than 0.05. In certain embodiments, the significance of the association of the mutation with reduced DRV susceptibility is indicated by a p-value calculated in Fisher's Exact test of less than 0.01.

### 5.4 Methods of Determining Increased Susceptibility to DRV

In certain aspects, the present invention provides methods for determining whether an HIV-1 has increased susceptibility to DRV. In general, the methods comprise detecting whether mutations significantly correlated with increased susceptibility to APV or DRV are present in the gene encoding protease or gag of the HIV-1, as demonstrated by the examples below.

Therefore, in certain embodiments, the invention provides a method for determining whether an HIV-1 is likely to have an increased susceptibility to darunavir, wherein if the HIV-1 exhibits an increased susceptibility to amprenavir, the HIV-1 also exhibits an increased susceptibility to darunavir. In one embodiment, the method comprises determining the susceptibility of the HIV-1 to amprenavir, wherein increased susceptibility to amprenavir correlates with increased susceptibility to darunavir, thereby determining whether the HIV-1 is likely to have an increased susceptibility to darunavir. In certain embodiments, increased susceptibility to amprenavir is determined by measuring in vitro the phenotypic sensitivity of the HIV-1 to amprenavir. In certain embodiments, increased susceptibility to amprenavir is determined by detecting, in a gene encoded by the HIV-1, the presence of one or more mutations associated with increased susceptibility to amprenavir.

### 5.4.1 Mutations in HIV-1 protease which correlate with increased susceptibility to APV

In certain embodiments, increased susceptibility to amprenavir is determined by detecting the presence of one or more mutations in at least one of codons 30, 43, 45, 50, 63, 64, 71, 77, 88 and 93 of the protease gene of the HIV-1, wherein the presence of the mutation(s) correlates with increased susceptibility to amprenavir, thereby determining that the HIV-1 is likely to have increased sensitivity to darunavir.

In certain embodiments, a mutation at codon 30 is detected. In certain embodiments, the mutation at codon 30 encodes asparagine (N). In certain embodiments, a mutation at codon 43 is detected. In certain embodiments, the mutation at codon 43 encodes arginine (R). In certain embodiments, a mutation at codon 45 is detected. In certain embodiments, the mutation at codon 45 encodes arginine (R). In certain embodiments, a mutation at codon 50 is detected. In certain embodiments, the mutation at codon 50 encodes leucine (L). In certain embodiments, a mutation at codon 63 is detected. In certain embodiments, the mutation at codon 63 encodes serine (S), alanine (A), or glutamine (Q). In certain embodiments, a mutation at codon 64 is detected. In certain embodiments, the mutation at codon 64 encodes valine (V). In certain embodiments, a mutation at codon 71 is detected. In certain embodiments, the mutation at codon 71 encodes threonine (T). In certain embodiments, a mutation at codon 77 is detected. In certain embodiments, the mutation at codon 77 encodes isoleucine (I). In certain embodiments, a mutation at codon 88 is detected. In certain embodiments, the mutation at codon 88 encodes aspartic acid (D) or serine (S). In certain embodiments, a mutation at codon 93 is detected. In certain embodiments, the mutation at codon 93 encodes leucine (L).

### 5.4.2 Mutations in HIV-1 Gag which Correlate with Increased Susceptibility to APV

In certain embodiments, increased susceptibility to amprenavir is determined by detecting the presence of one or more mutations in at least one of codons 441, 451, 486, 498 and 499 of the gag gene of the HIV-1, wherein the presence of the mutation(s) correlates with increased susceptibility to amprenavir, thereby determining that the HIV-1 is likely to have increased sensitivity to darunavir.

In certain embodiments, a mutation at codon 441 is detected. In certain embodiments, the mutation at codon 441 encodes tyrosine (Y). In certain embodiments, a mutation at codon 451 is detected. In certain embodiments, the mutation at codon 451 encodes asparagines (N). In certain embodiments, a mutation at codon 486 is detected. In certain embodiments, the mutation at codon 486 encodes serine (S). In certain embodiments, a mutation at codon 498 is detected. In certain embodiments, the mutation at codon 498 encodes leucine (L). In certain embodiments, a mutation at codon 499 is detected. In certain embodiments, the mutation at codon 499 encodes leucine (L).

### 5.4.3 Mutations in HIV-1 Protease which Correlate with Increased Susceptibility to DRV

In another aspect, the invention provides a method for determining whether an HIV-1 is likely to have an increased susceptibility to darunavir, comprising detecting whether an HIV-1 protease mutation is present in at least one of codons 20, 30, 36, 41, 43, 45, 50, 63, 64, 65, 70, 71, 74, 75, 77, 82, 88 and 93 of the HIV-1, wherein the presence of the mutation(s) correlates with increased susceptibility to treatment with darunavir, thereby determining whether the HIV-1 is likely to have an increased susceptibility to darunavir.

In certain embodiments, a mutation at codon 43 is detected. In certain embodiments, the mutation at codon 43 is arginine (R). In certain embodiments, a mutation at codon 45 is detected In certain embodiments, the mutation at codon 45 is arginine (R). In certain embodiments, a mutation at codon 63 is detected. In certain embodiments, the mutation at codon 63 is glutamine (Q).

In certain embodiments, the HIV-1 is determined to have an increased susceptibility to darunavir by detecting an HIV-1 protease mutation, wherein the mutation is significantly associated with increased susceptibility to DRV, as indicated in Table 1. In certain embodiments, the significance of the association of the mutation with increased DRV susceptibility is indicated by an odds ratio less than 1. In certain embodiments, the mutation has an odds ratio less than 0.5. In certain embodiments, the mutation has an odds ratio less than 0.3. In certain embodiments, the significance of the association of the mutation with reduced DRV susceptibility is indicated by a p-value calculated in Fisher's Exact test (including the Benjamini correction for multiple comparisons) of less than 0.05. In certain embodiments, the significance of the association of the mutation with reduced DRV susceptibility is indicated by a p-value calculated in Fisher's Exact test of less than 0.01.

In certain embodiments, the methods further comprise detecting whether a mutation in at least one of codons 20, 30, 36, 41, 43, 45, 50, 63, 64, 65, 70, 71, 74, 75, 77, 82, 88 and 93 is present in combination with at least one of codons 30, 50 or 88 of the HIV-1. In certain embodiments, a combination of mutations is detected comprising a mutation at codon 30. In certain embodiments, the mutation at codon 30 encodes asparagine (N). In certain embodiments, a combination of mutations is detected comprising a mutation at codon 50. In certain embodiments, the mutation at codon 50 encodes leucine (L). In certain embodiments, a combination of mutations is detected comprising a mutation at codon 88. In certain embodiments, the mutation at codon 88 encodes aspartic acid (D) or serine (S).

### 5.4.4 Mutations in HIV-1 Gag which Correlate with Increased Susceptibility to DRV

In another aspect, the invention provides a method for determining whether an HIV-1 is likely to have an increased susceptibility to darunavir, comprising detecting whether an HIV-1 gag mutation is present in at least one of codons 437, 439, 441, 442, 451, 475, 480, 482, 483, 486, 498 and 499 of the HIV-1, wherein the presence of the mutation(s) correlates with increased susceptibility to treatment with darunavir, thereby determining whether the HIV-1 is likely to have an increased susceptibility to darunavir.

In certain embodiments, a mutation at codon 437 is detected. In certain embodiments, the mutation at codon 437 encodes leucine (L). In certain embodiments, a mutation at codon 439 is detected. In certain embodiments, the mutation at codon 439 encodes serine (S). In certain embodiments, a mutation at codon 480 is detected. In certain embodiments, the mutation at codon 480 results in deletion of the codon. In certain embodiments, a mutation at codon 483 is detected. In certain embodiments, the mutation at codon 483 encodes arginine (R). In certain embodiments, a mutation at codon 498 is detected. In certain embodiments, the mutation at codon 498 encodes leucine (L).

In certain embodiments, the HIV-1 is determined to have an increased susceptibility to darunavir by detecting an HIV-1 gag mutation, wherein the mutation is significantly associated with increased susceptibility to DRV, as indicated in Table 1. In certain embodiments, the significance of the association of the mutation with increased DRV susceptibility is indicated by an odds ratio less than 1. In certain embodiments, the mutation has an odds ratio less than 0.5. In certain embodiments, the mutation has an odds ratio less than 0.3. In certain embodiments, the significance of the association of the mutation with reduced DRV susceptibility is indicated by a p-value calculated in Fisher's Exact test of less than 0.05. In certain embodiments, the significance of the association of the mutation with reduced DRV susceptibility is indicated by a p-value calculated in Fisher's Exact test of less than 0.01.

### 5.5 Measuring Altered Susceptibility of HIV-1 to APV and/or DRV

Any method known in the art can be used to determine a viral drug susceptibility phenotype, without limitation. See e.g., U.S. Pat. Nos. 5,837,464 and 6,242,187, each of which is hereby incorporated by reference in its entirety.

In certain embodiments, the phenotypic analysis is performed using recombinant virus assays (“RVAs”). RVAs use virus stocks generated by homologous recombination between viral vectors and viral gene sequences, amplified from the patient virus. In certain embodiments, the viral vector is a HIV vector and the viral gene sequences are protease and/or reverse transcriptase and/or gag sequences.

In preferred embodiments, the phenotypic analysis of altered susceptibility to APV and/or DRV is performed using PHENOSENSE™ (Monogram Biosciences, South San Francisco, Calif.). See Petropoulos et al., 2000, Antimicrob. Agents Chemother. 44:920-928; U.S. Pat. Nos. 5,837,464 and 6,242,187. PHENOSENSE™ is a phenotypic assay that achieves the benefits of phenotypic testing and overcomes the drawbacks of previous assays. Because the assay has been automated, PHENOSENSE™ provides high throughput methods under controlled conditions for determining APV and/or DRV resistance, susceptibility, or hypersusceptibility of a large number of individual viral isolates.

The result is an assay that can quickly and accurately define both the replication capacity and the susceptibility profile of a patient's HIV (or other virus) isolates to all currently available antiretroviral drugs, including the PIs APV and DRV. PHENOSENSE™ can obtain results with only one round of viral replication, thereby avoiding selection of subpopulations of virus that can occur during preparation of viral stocks required for assays that rely on fully infectious virus. Further, the results are both quantitative, measuring varying degrees of replication capacity or antiviral resistance or susceptibility, and sensitive, as the test can be performed on blood specimens with a viral load of about 500 copies/mL or above and can detect minority populations of some drug resistant virus at concentrations of 10% or less of total viral population. Finally, the replication capacity and antiviral drug resistance results are reproducible and can vary by less than about 0.25 logs in about 95% of the assays performed.

PHENOSENSE™ can be used with nucleic acids from amplified viral gene sequences. As discussed below, the nucleic acid can be amplified from any sample known by one of skill in the art to contain a viral gene sequence, without limitation. For example, the sample can be a sample from a human or an animal infected with the virus or a sample from a culture of viral cells. In certain embodiments, the viral sample comprises a genetically modified laboratory strain. In other embodiments, the viral sample comprises a wild-type isolate.

A resistance test vector (“RTV”) can then be constructed by incorporating the amplified viral gene sequences into a replication defective viral vector by using any method known in the art of incorporating gene sequences into a vector. In one embodiment, restrictions enzymes and conventional cloning methods are used. See Sambrook et al., 2001, Molecular Cloning: A Laboratory Manual, Cold Spring Harbor Laboratory, 3rd ed., NY; and Ausubel et al., 1989, Current Protocols in Molecular Biology, Greene Publishing Associates and Wiley Interscience, NY. In a preferred embodiment, ApaI and PinAI restriction enzymes are used. Preferably, the replication defective viral vector is the indicator gene viral vector (“IGVV”). In a preferred embodiment, the viral vector contains a means for detecting replication of the RTV. Preferably, the viral vector contains a luciferase expression cassette.

The assay can be performed by first co-transfecting host cells with RTV DNA and a plasmid that expresses the envelope proteins of another retrovirus, for example, amphotropic murine leukemia virus (MLV). Following transfection, viral particles can be harvested from the cell culture and used to infect fresh target cells in the presence of varying amounts of anti-viral drug(s). The completion of a single round of viral replication in the fresh target cells can be detected by the means for detecting replication contained in the vector. In a preferred embodiment, the completion of a single round of viral replication results in the production of luciferase. By monitoring the amount of, e.g., luciferase activity in the presence of the varying amounts of antiviral drug(s), a resistance curve can be constructed by plotting luciferase activity versus drug concentration. The susceptibility of an HIV, or population of HIV, can be determined by measuring the concentration of antiviral drug at which the luciferase activity detected is half-maximal. This number, the IC₅₀, provides a standard and convenient measure of drug resistance.

In preferred embodiments, PHENOSENSE™ is used to evaluate the APV and/or DRV susceptibility phenotype of HIV 1. In other embodiments, PHENOSENSE™ is used to evaluate the APV and/or DRV susceptibility phenotype of HIV 2. In certain embodiments, the HIV-1 strain that is evaluated is a wild-type isolate of HIV-1. In other embodiments, the HIV-1 strain that is evaluated is a mutant strain of HIV-1. In certain embodiments, such mutant strains can be isolated from patients. In other embodiments, the mutant strains can be constructed by site-directed mutagenesis or other equivalent techniques known to one of skill in the art. In still other embodiments, the mutant strains can be isolated from cell culture. The cultures can comprise multiple passages through cell culture in the presence of antiviral compounds to select for mutations that accumulate in culture in the presence of such compounds.

In one embodiment, viral nucleic acid, for example, HIV 1 RNA is extracted from plasma samples, and a fragment of, or entire viral genes can be amplified by methods such as, but not limited to PCR. See, e.g., Hertogs et al., 1998, Antimicrob Agents Chemother 42(2):269 76. In one example, a 2.2 kb fragment containing the entire HIV 1 PR and RT coding sequence is amplified by nested reverse transcription PCR. The pool of amplified nucleic acid, for example, the PR RT coding sequences, is then cotransfected into a host cell such as CD4+T lymphocytes (MT4) with the pGEMT3deltaPRT plasmid from which most of the PR (codons 10 to 99) and RT (codons 1 to 482) sequences are deleted. Homologous recombination leads to the generation of chimeric viruses containing viral coding sequences, such as the PR and RT coding sequences derived from HIV 1 RNA in plasma. The replication capacities or antiviral drug resistance phenotypes of the chimeric viruses can be determined by any cell viability assay known in the art, and compared to replication capacities or antiviral drug susceptibilities of a statistically significant number of individual viral isolates to assess whether a virus has altered replication capacity or is resistant or hypersusceptible to the antiviral drug. For example, an MT4 cell 3 (4,5 dimethylthiazol 2 yl) 2,5 diphenyltetrazolium bromide based cell viability assay can be used in an automated system that allows high sample throughput.

In another embodiment, competition assays can be used to assess replication capacity of one viral strain relative to another viral strain. For example, two infectious viral strains can be co-cultivated together in the same culture medium. See, e.g., Lu et al., 2001, JAIDS 27:7-13, which is incorporated by reference in its entirety. By monitoring the course of each viral strain's growth, the fitness of one strain relative to the other can be determined. By measuring many viruses' fitness relative to a single reference virus, an objective measure of each strain's fitness can be determined.

Other assays for evaluating the phenotypic susceptibility of a virus to anti-viral drugs known to one of skill in the art can be adapted to determine replication capacity or to determine antiviral drug susceptibility or resistance. See, e.g., Shi and Mellors, 1997, Antimicrob Agents Chemother. 41(12):2781-85; Gervaix et al., 1997, Proc Natl Acad Sci U.S.A. 94(9):4653-8; Race et al., 1999, AIDS 13:2061-2068, incorporated herein by reference in their entireties.

In addition, the phenotypic assays described above can also be used to determine the effectiveness of candidate compounds. Generally, such methods comprise performing the phenotypic assay in the presence and absence of the candidate compound, wherein the difference in activity or expression of the indicator gene indicates the effectiveness of the candidate compound. Advantageously, the methods can be performed in the presence of a mutation associated with altered susceptibility to APV and/or DRV as disclosed herein. By performing such assays in the presence of such mutations, candidate compounds can be identified that have beneficial interactions with the PIs to which the virus has altered susceptibility. In certain embodiments, the candidate compound will have an additive effect on viral inhibition with the PI. In preferred embodiments, the candidate compound will have a synergistic effect on viral inhibition with the PI. Further guidance may be found in performing the assays to determine the effectiveness of candidate compounds in Petropoulos et al., 2000, Antimicrob. Agents Chemother. 44:920-928; and U.S. Pat. Nos. 5,837,464 and 6,242,187.

### 5.5.1 Detecting the Presence or Absence of Mutations in a Virus

The presence or absence of a mutation associated with altered susceptibility according to the present invention in a virus can be determined by any means known in the art for detecting a mutation. The mutation can be detected in the viral gene that encodes a particular protein, or in the protein itself, i.e., in the amino acid sequence of the protein.

In one embodiment, the mutation is in the viral genome. Such a mutation can be in, for example, a gene encoding a viral protein, in a genetic element such as a cis or trans acting regulatory sequence of a gene encoding a viral protein, an intergenic sequence, or an intron sequence. The mutation can affect any aspect of the structure, function, replication or environment of the virus that changes its susceptibility to an anti-viral treatment and/or its replication capacity. In one embodiment, the mutation is in a gene encoding a viral protein that is the target of an currently available anti-viral treatment. In other embodiments, the mutation is in a gene or other genetic element that is not the target of a currently-available anti-viral treatment.

A mutation within a viral gene can be detected by utilizing any suitable technique known to one of skill in the art without limitation. Viral DNA or RNA can be used as the starting point for such assay techniques, and may be isolated according to standard procedures which are well known to those of skill in the art.

The detection of a mutation in specific nucleic acid sequences, such as in a particular region of a viral gene, can be accomplished by a variety of methods including, but not limited to, restriction-fragment-length-polymorphism detection based on allele specific restriction-endonuclease cleavage (Kan and Dozy, 1978, Lancet ii:910-912), mismatch-repair detection (Faham and Cox, 1995, Genome Res 5:474-482), binding of MutS protein (Wagner et al., 1995, Nucl Acids Res 23:3944-3948), denaturing-gradient gel electrophoresis (Fisher et al., 1983, Proc. Natl. Acad. Sci. U.S.A. 80:1579 83), single-strand-conformation-polymorphism detection (Orita et al., 1983, Genomics 5:874-879), RNAase cleavage at mismatched base-pairs (Myers et al., 1985, Science 230:1242), chemical (Cotton et al., 1988, Proc. Natl. Acad. Sci. U.S.A. 85:4397-4401) or enzymatic (Youil et al., 1995, Proc. Natl. Acad. Sci. U.S.A. 92:87-91) cleavage of heteroduplex DNA, methods based on oligonucleotide-specific primer extension (Syvänen et al., 1990, Genomics 8:684-692), genetic bit analysis (Nikiforov et al., 1994, Nucl Acids Res 22:4167-4175), oligonucleotide-ligation assay (Landegren et al., 1988, Science 241:1077), oligonucleotide-specific ligation chain reaction (“LCR”) (Barrany, 1991, Proc. Natl. Acad. Sci. U.S.A. 88:189-193), gap-LCR (Abravaya et al., 1995, Nucl Acids Res 23:675-682), radioactive or fluorescent DNA sequencing using standard procedures well known in the art, and peptide nucleic acid (PNA) assays (Drum et al., 1993, Nucl. Acids Res. 21:5332-5356; Thiede et al., 1996, Nucl. Acids Res. 24:983-984).

In addition, viral DNA or RNA may be used in hybridization or amplification assays to detect abnormalities involving gene structure, including point mutations, insertions, deletions and genomic rearrangements. Such assays may include, but are not limited to, Southern analyses (Southern, 1975, J. Mol. Biol. 98:503 517), single stranded conformational polymorphism analyses (SSCP) (Orita et al., 1989, Proc. Natl. Acad. Sci. USA 86:2766 2770), and PCR analyses (U.S. Pat. Nos. 4,683,202; 4,683,195; 4,800,159; and 4,965,188; PCR Strategies, 1995 Innis et al. (eds.), Academic Press, Inc.).

Such diagnostic methods for the detection of a gene specific mutation can involve for example, contacting and incubating the viral nucleic acids with one or more labeled nucleic acid reagents including recombinant DNA molecules, cloned genes or degenerate variants thereof, under conditions favorable for the specific annealing of these reagents to their complementary sequences. Preferably, the lengths of these nucleic acid reagents are at least 15 to 30 nucleotides. After incubation, all non-annealed nucleic acids are removed from the nucleic acid molecule hybrid. The presence of nucleic acids which have hybridized, if any such molecules exist, is then detected. Using such a detection scheme, the nucleic acid from the virus can be immobilized, for example, to a solid support such as a membrane, or a plastic surface such as that on a microtiter plate or polystyrene beads. In this case, after incubation, non annealed, labeled nucleic acid reagents of the type described above are easily removed. Detection of the remaining, annealed, labeled nucleic acid reagents is accomplished using standard techniques well known to those in the art. The gene sequences to which the nucleic acid reagents have annealed can be compared to the annealing pattern expected from a normal gene sequence in order to determine whether a gene mutation is present.

These techniques can easily be adapted to provide high-throughput methods for detecting mutations in viral genomes. For example, a gene array from Affymetrix (Affymetrix, Inc., Sunnyvale, Calif.) can be used to rapidly identify genotypes of a large number of individual viruses. Affymetrix gene arrays, and methods of making and using such arrays, are described in, for example, U.S. Pat. Nos. 6,551,784, 6,548,257, 6,505,125, 6,489,114, 6,451,536, 6,410,229, 6,391,550, 6,379,895, 6,355,432, 6,342,355, 6,333,155, 6,308,170, 6,291,183, 6,287,850, 6,261,776, 6,225,625, 6,197,506, 6,168,948, 6,156,501, 6,141,096, 6,040,138, 6,022,963, 5,919,523, 5,837,832, 5,744,305, 5,834,758, and 5,631,734, each of which is hereby incorporated by reference in its entirety.

In addition, Ausubel et al., eds., Current Protocols in Molecular Biology, 2002, Vol. 4, Unit 25B, Ch. 22, which is hereby incorporated by reference in its entirety, provides further guidance on construction and use of a gene array for determining the genotypes of a large number of viral isolates. Finally, U.S. Pat. Nos. 6,670,124; 6,617,112; 6,309,823; 6,284,465; and 5,723,320, each of which is incorporated by reference in its entirety, describe related array technologies that can readily be adapted for rapid identification of a large number of viral genotypes by one of skill in the art.

Alternative diagnostic methods for the detection of gene specific nucleic acid molecules may involve their amplification, e.g., by PCR (U.S. Pat. Nos. 4,683,202; 4,683,195; 4,800,159; and 4,965,188; PCR Strategies, 1995 Innis et al. (eds.), Academic Press, Inc.), followed by the detection of the amplified molecules using techniques well known to those of skill in the art. The resulting amplified sequences can be compared to those which would be expected if the nucleic acid being amplified contained only normal copies of the respective gene in order to determine whether a gene mutation exists.

Additionally, the nucleic acid can be sequenced by any sequencing method known in the art. For example, the viral DNA can be sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499. See also the techniques described in Sambrook et al., 2001, Molecular Cloning: A Laboratory Manual, Cold Spring Harbor Laboratory, 3rd ed., NY; and Ausubel et al., 1989, Current Protocols in Molecular Biology, Greene Publishing Associates and Wiley Interscience, NY.

Antibodies directed against the viral gene products, i.e., viral proteins or viral peptide fragments can also be used to detect mutations in the viral proteins. Alternatively, the viral protein or peptide fragments of interest can be sequenced by any sequencing method known in the art in order to yield the amino acid sequence of the protein of interest. An example of such a method is the Edman degradation method which can be used to sequence small proteins or polypeptides. Larger proteins can be initially cleaved by chemical or enzymatic reagents known in the art, for example, cyanogen bromide, hydroxylamine, trypsin or chymotrypsin, and then sequenced by the Edman degradation method.

### 5.5.2 Correlating Mutations with Altered APV and/or DRV Susceptibility

Any method known in the art can be used to determine whether a mutation is correlated with altered APV and/or DRV susceptibility. In one embodiment, univariate analysis is used to identify mutations correlated with altered APV and/or DRV susceptibility. Univariate analysis yields P values that indicate the statistical significance of the correlation. In such embodiments, the smaller the P value, the more significant the measurement. Preferably the P values, after correction for false discovery rate due to multiple comparisons (Benjamini, Y., and Hochberg, Y. (1995). Controlling the false discovery rate: a practical and powerful approach to multiple testing._Journal of the Royal Statistical Society Series_B, 57, 289-300), will be less than 0.05. More preferably, P values will be less than 0.01. Even more preferably, the P value will be less than 0.005. P values can be calculated by any means known to one of skill in the art. In one embodiment, P values are calculated using Fisher's Exact Test. In another embodiment, P values can be calculated with Student's t-test. See, e.g., David Freedman, Robert Pisani & Roger Purves, 1980, STATISTICS, W. W. Norton, New York. In certain embodiments, P values can be calculated with both Fisher's Exact Test and Student's t-test. In such embodiments, P values calculated with both tests are preferably less than 0.05. However, a correlation with a P value that is less than 0.10 in one test but less than 0.05 in another test can still be considered to be a marginally significant correlation. Such mutations are suitable for further analysis with, for example, multivariate analysis. Alternatively, further univariate analysis can be performed on a larger sample set to confirm the significance of the correlation.

Further, an odds ratio can be calculated to determine whether a mutation correlates with altered APV and/or DRV susceptibility. Generally, calculation of odds ratios depends on dividing the percentage of virus that are identified as having altered APV and/or DRV susceptibility and that comprise a particular mutation or mutations by the percentage of virus identified as not having altered APV and/or DRV susceptibility with the same mutation or mutations. In certain embodiments, an odds ratio that is greater than one indicates that the mutation correlates with reduced susceptibility. In certain embodiments, an odds ratio that is greater than two indicates that the mutation correlates with reduced susceptibility. In certain embodiments, an odds ratio that is greater than three indicates that the mutation correlates with reduced susceptibility. In certain embodiments, an odds ratio that is less than one indicates that the mutation correlates with increased susceptibility. In certain embodiments, an odds ratio that is less than 0.5 indicates that the mutation correlates with increased susceptibility. In certain embodiments, an odds ratio that is less than 0.3 indicates that the mutation correlates with increased susceptibility.

In yet another embodiment, multivariate analysis can be used to determine whether a mutation correlates with altered APV and/or DRV susceptibility. Any multivariate analysis known by one of skill in the art to be useful in calculating such a correlation can be used, without limitation. In certain embodiments, a statistically significant number of virus's resistance or susceptibility phenotypes, e.g., IC50, can be determined. These IC50 values can then be divided into groups that correspond to percentiles of the set of IC50 values observed.

After assigning each virus's IC50 value to the appropriate group, the genotype of that virus can be assigned to that group. By performing this method for all viral isolates, the number of instances of a particular mutation in a given percentile of altered APV and/or DRV susceptibility can be observed. This allows the skilled practitioner to identify mutations that correlate with altered APV and/or DRV susceptibility.

Finally, in yet another embodiment, regression analysis can be performed to identify mutations that best predict altered APV and/or DRV susceptibility. In such embodiments, regression analysis is performed on a statistically significant number of viral isolates for which genotypes and altered APV and/or DRV susceptibility phenotypes have been determined. The analysis then identifies which mutations appear to best predict, e.g., most strongly correlate with, altered APV and/or DRV susceptibility. Such analysis can then be used to construct rules for predicting altered APV and/or DRV susceptibility based upon knowledge of the genotype of a particular virus, described below. In certain embodiments, software such as, for example, CART 5.0, Prism 4.0, or Insightful Miner 3.0 can be used to perform the analysis that identifies the mutations that appear to best predict altered APV and/or DRV susceptibility.

### 5.5.3 Computer-Implemented Methods for Determining Altered APV and/or DRV Susceptibility, and Articles Related Thereto

In another aspect, the present invention provides computer-implemented methods for determining that an HIV-1 has altered APV and/or DRV susceptibility. In such embodiments, the methods of the invention are adapted to take advantage of the processing power of modern computers. One of skill in the art can readily adapt the methods in such a manner.

Reduced APV and/or DRV Susceptibility

Therefore, in certain embodiments, the invention provides a computer-implemented method for determining that an HIV-1 has reduced susceptibility to DRV, comprising inputting genetic information into a memory system of a computer, wherein the genetic information indicates that the HIV-1 has a gene encoding protease with a mutation in at least one of codons 4, 10, 11, 12, 13, 15, 16, 19, 20, 22, 23, 24, 32, 34, 35, 36, 37, 43, 46, 47, 50, 53, 54, 55, 58, 60, 61, 62, 63, 66, 67, 69, 71, 72, 73, 74, 76, 79, 82, 84, 85, 89, 90, 91, 92 and 95, inputting a correlation between the presence of the mutations and reduced susceptibility to APV into the memory system of the computer, and determining that the HIV-1 has reduced susceptibility to DRV.

In certain embodiments, the genetic information indicates that the HIV-1 has a mutation at codon 4. In certain embodiments, the mutation at codon 4 encodes alanine (A), proline (P) or serine (S). In certain embodiments, the genetic information indicates that the HIV-1 has a mutation at codon 10. In certain embodiments, the mutation at codon 10 encodes phenylalanine (F), valine (V) or isoleucine (I). In certain embodiments, the genetic information indicates that the HIV-1 has a mutation at codon 11. In certain embodiments, the mutation at codon 11 leucine (L) or isoleucine (I). In certain embodiments, the genetic information indicates that the HIV-1 has a mutation at codon 12. In certain embodiments, the mutation at codon 12 encodes alanine proline (P). In certain embodiments, the genetic information indicates that the HIV-1 has a mutation at codon 13. In certain embodiments, the mutation at codon 13 encodes valine (V). In certain embodiments, the genetic information indicates that the HIV-1 has a mutation at codon 15. In certain embodiments, the mutation at codon 15 encodes valine (V). In certain embodiments, the genetic information indicates that the HIV-1 has a mutation at codon 16. In certain embodiments, the mutation at codon 16 encodes alanine (A). In certain embodiments, the genetic information indicates that the HIV-1 has a mutation at codon 19. In certain embodiments, the mutation at codon 19 encodes proline (P). In certain embodiments, the genetic information indicates that the HIV-1 has a mutation at codon 20. In certain embodiments, the mutation at codon 20 encodes valine (V), arginine (R) or threonine (T). In certain embodiments, the genetic information indicates that the HIV-1 has a mutation at codon 22. In certain embodiments, the mutation at codon 22 encodes valine (V). In certain embodiments, the genetic information indicates that the HIV-1 has a mutation at codon 23. In certain embodiments, the mutation at codon 23 encodes isoleucine (I). In certain embodiments, the genetic information indicates that the HIV-1 has a mutation at codon 24. In certain embodiments, the mutation at codon 24 encodes phenylalanine (F). In certain embodiments, the genetic information indicates that the HIV-1 has a mutation at codon 32. In certain embodiments, the mutation at codon 32 encodes isoleucine (I). In certain embodiments, the genetic information indicates that the HIV-1 has a mutation at codon 33. In certain embodiments, the mutation at codon 33 encodes phenylalanine (F). In certain embodiments, the genetic information indicates that the HIV-1 has a mutation at codon 34. In certain embodiments, the mutation at codon 34 encodes glutamine (Q). In certain embodiments, the genetic information indicates that the HIV-1 has a mutation at codon 35. In certain embodiments, the mutation at codon 35 encodes asparagine (N) or aspartic acid (D). In certain embodiments, the genetic information indicates that the HIV-1 has a mutation at codon 36. In certain embodiments, the mutation at codon 36 encodes leucine (L) or isoleucine (I). In certain embodiments, the genetic information indicates that the HIV-1 has a mutation at codon 37. In certain embodiments, the mutation at codon 37 encodes aspartic acid (D). In certain embodiments, the genetic information indicates that the HIV-1 has a mutation at codon 43. In certain embodiments, the mutation at codon 43 encodes threonine (T). In certain embodiments, the genetic information indicates that the HIV-1 has a mutation at codon 46. In certain embodiments, the mutation at codon 46 encodes isoleucine (I) or leucine (L). In certain embodiments, the genetic information indicates that the HIV-1 has a mutation at codon 47. In certain embodiments, the mutation at codon 47 encodes valine (V). In certain embodiments, the genetic information indicates that the HIV-1 has a mutation at codon 50. In certain embodiments, the mutation at codon 50 encodes valine (V). In certain embodiments, the genetic information indicates that the HIV-1 has a mutation at codon 53. In certain embodiments, the mutation at codon 53 encodes tyrosine (Y) or leucine (L). In certain embodiments, the genetic information indicates that the HIV-1 has a mutation at codon 54. In certain embodiments, the mutation at codon 54 encodes methionine (M), leucine (L), serine (S), threonine (T), alanine (A) or valine (V). In certain embodiments, the genetic information indicates that the HIV-1 has a mutation at codon 55. In certain embodiments, the mutation at codon 55 encodes asparagines (N) or arginine (R). In certain embodiments, the genetic information indicates that the HIV-1 has a mutation at codon 58. In certain embodiments, the mutation at codon 58 encodes glutamic acid (E). In certain embodiments, the genetic information indicates that the HIV-1 has a mutation at codon 60. In certain embodiments, the mutation at codon 60 encodes glutamic acid (E). In certain embodiments, the genetic information indicates that the HIV-1 has a mutation at codon 61. In certain embodiments, the mutation at codon 61 encodes asparagine (N). In certain embodiments, the genetic information indicates that the HIV-1 has a mutation at codon 62. In certain embodiments, the mutation at codon 62 encodes valine (V). In certain embodiments, the genetic information indicates that the HIV-1 has a mutation at codon 63. In certain embodiments, the mutation at codon 63 encodes proline (P). In certain embodiments, the genetic information indicates that the HIV-1 has a mutation at codon 66. In certain embodiments, the mutation at codon 66 encodes valine (V) or phenylalanine (F). In certain embodiments, the genetic information indicates that the HIV-1 has a mutation at codon 67. In certain embodiments, the mutation at codon 67 encodes phenylalanine (F). In certain embodiments, the genetic information indicates that the HIV-1 has a mutation at codon 69. In certain embodiments, the mutation at codon 69 encodes arginine (R). In certain embodiments, the genetic information indicates that the HIV-1 has a mutation at codon 71. In certain embodiments, the mutation at codon 71 encodes leucine (L), isoleucine (I) or valine (V). In certain embodiments, the genetic information indicates that the HIV-1 has a mutation at codon 72. In certain embodiments, the mutation at codon 72 encodes leucine (L) or valine (V). In certain embodiments, the genetic information indicates that the HIV-1 has a mutation at codon 73. In certain embodiments, the mutation at codon 73 encodes alanine (A), threonine (T), cysteine (C) or serine (S). In certain embodiments, the genetic information indicates that the HIV-1 has a mutation at codon 74. In certain embodiments, the mutation at codon 74 encodes proline (P). In certain embodiments, the genetic information indicates that the HIV-1 has a mutation at codon 76. In certain embodiments, the mutation at codon 76 encodes valine (V). In certain embodiments, the genetic information indicates that the HIV-1 has a mutation at codon 79. In certain embodiments, the mutation at codon 79 encodes alanine (A) or serine (S). In certain embodiments, the genetic information indicates that the HIV-1 has a mutation at codon 82. In certain embodiments, the mutation at codon 82 encodes phenylalanine (F), leucine (L), cysteine (C), isoleucine (I), threonine (T) or alanine (A). In certain embodiments, the genetic information indicates that the HIV-1 has a mutation at codon 84. In certain embodiments, the mutation at codon 84 encodes valine (V). In certain embodiments, the genetic information indicates that the HIV-1 has a mutation at codon 85. In certain embodiments, the mutation at codon 85 encodes valine (V). In certain embodiments, the genetic information indicates that the HIV-1 has a mutation at codon 89. In certain embodiments, the mutation at codon 89 encodes valine (V), isoleucine (I) or methionine (M). In certain embodiments, the genetic information indicates that the HIV-1 has a mutation at codon 90. In certain embodiments, the mutation at codon 90 encodes methionine (M). In certain embodiments, the genetic information indicates that the HIV-1 has a mutation at codon 91. In certain embodiments, the mutation at codon 91 encodes serine (S). In certain embodiments, the genetic information indicates that the HIV-1 has a mutation at codon 92. In certain embodiments, the mutation at codon 92 encodes lysine (K). In certain embodiments, the genetic information indicates that the HIV-1 has a mutation at codon 95. In certain embodiments, the mutation at codon 95 encodes valine (V).

In certain embodiments, the invention provides a computer-implemented method for determining that an HIV-1 has reduced susceptibility to DRV, comprising inputting genetic information into a memory system of a computer, wherein the genetic information indicates that the HIV-1 has a gene encoding gag with a mutation in at least one of codons 423, 425, 428, 431, 437, 441, 449, 451, 452, 453, 459, 462, 463, 465, 467, 468, 479 and 488, inputting a correlation between the presence of the mutations and reduced susceptibility to APV into the memory system of the computer, and determining that the HIV-1 has reduced susceptibility to DRV.

In certain embodiments, the genetic information indicates that the HIV-1 has a mutation at codon 423. In certain embodiments, the mutation at codon 423 encodes isoleucine (I). In certain embodiments, the genetic information indicates that the HIV-1 has a mutation at codon 425. In certain embodiments, the mutation at codon 425 encodes glutamic acid (E). In certain embodiments, the genetic information indicates that the HIV-1 has a mutation at codon 428. In certain embodiments, the mutation at codon 428 encodes aspartic acid (D). In certain embodiments, the genetic information indicates that the HIV-1 has a mutation at codon 431. In certain embodiments, the mutation at codon 431 encodes isoleucine (I) or valine (V). In certain embodiments, the genetic information indicates that the HIV-1 has a mutation at codon 437. In certain embodiments, the mutation at codon 437 encodes valine (V). In certain embodiments, the genetic information indicates that the HIV-1 has a mutation at codon 441. In certain embodiments, the mutation at codon 441 encodes glutamine (Q). In certain embodiments, the genetic information indicates that the HIV-1 has a mutation at codon 449. In certain embodiments, the mutation at codon 449 encodes valine (V) or phenylalanine (F). In certain embodiments, the genetic information indicates that the HIV-1 has a mutation at codon 451. In certain embodiments, the mutation at codon 451 encodes threonine (T). In certain embodiments, the genetic information indicates that the HIV-1 has a mutation at codon 452. In certain embodiments, the mutation at codon 452 encodes serine (S) or lysine (K). In certain embodiments, the genetic information indicates that the HIV-1 has a mutation at codon 453. In certain embodiments, the mutation at codon 453 encodes valine (V), isoleucine (I) or leucine (L). In certain embodiments, the genetic information indicates that the HIV-1 has a mutation at codon 459. In certain embodiments, the mutation at codon 459 encodes threonine (T). In certain embodiments, the genetic information indicates that the HIV-1 has a mutation at codon 462. In certain embodiments, the mutation at codon 462 encodes asparagine (N). In certain embodiments, the genetic information indicates that the HIV-1 has a mutation at codon 463. In certain embodiments, the mutation at codon 463 encodes serine (S), valine (V) or leucine (L). In certain embodiments, the genetic information indicates that the HIV-1 has a mutation at codon 465. In certain embodiments, the mutation at codon 465 encodes serine (S) or leucine (L). In certain embodiments, the genetic information indicates that the HIV-1 has a mutation at codon 467. In certain embodiments, the mutation at codon 467 encodes lysine (K). In certain embodiments, the genetic information indicates that the HIV-1 has a mutation at codon 468. In certain embodiments, the mutation at codon 468 encodes lysine (K). In certain embodiments, the genetic information indicates that the HIV-1 has a mutation at codon 479. In certain embodiments, the mutation at codon 479 encodes threonine (T). In certain embodiments, the genetic information indicates that the HIV-1 has a mutation at codon 488. In certain embodiments, the mutation at codon 488 encodes alanine (A).

In certain embodiments, the invention provides a computer-implemented method for determining that an HIV-1 has reduced susceptibility to DRV, comprising inputting genetic information into a memory system of a computer, wherein the genetic information indicates that the HIV-1 has a gene encoding protease with a mutation in at least one of codons 4, 10, 12, 13, 15, 16, 18, 19, 20, 22, 23, 24, 34, 35, 36, 37, 43, 46, 48, 53, 55, 57, 58, 60, 61, 62, 63, 66, 67, 69, 71, 72, 74, 79, 82, 83, 85, 90, 91, 92, and 95, inputting a correlation between the presence of the mutations and reduced susceptibility to DRV into the memory system of the computer, and determining that the HIV-1 has reduced susceptibility to DRV.

In certain embodiments, the genetic information indicates that the HIV-1 has a mutation at codon 4. In certain embodiments, the mutation at codon 4 encodes alanine (A) or proline (P). In certain embodiments, the genetic information indicates that the HIV-1 has a mutation at codon 16. In certain embodiments, the mutation at codon 16 encodes alanine (A). In certain embodiments, the genetic information indicates that the HIV-1 has a mutation at codon 19. In certain embodiments, the mutation at codon 19 encodes proline (P). In certain embodiments, the genetic information indicates that the HIV-1 has a mutation at codon 20. In certain embodiments, the mutation at codon 20 encodes valine (V). In certain embodiments, the genetic information indicates that the HIV-1 has a mutation at codon 22. In certain embodiments, the mutation at codon 22 encodes valine (V). In certain embodiments, the genetic information indicates that the HIV-1 has a mutation at codon 24. In certain embodiments, the mutation at codon 24 encodes phenylalanine (F). In certain embodiments, the genetic information indicates that the HIV-1 has a mutation at codon 34. In certain embodiments, the mutation at codon 34 encodes glutamine (Q). In certain embodiments, the genetic information indicates that the HIV-1 has a mutation at codon 43. In certain embodiments, the mutation at codon 43 encodes threonine (T). In certain embodiments, the genetic information indicates that the HIV-1 has a mutation at codon 53. In certain embodiments, the mutation at codon 53 encodes tyrosine (Y). In certain embodiments, the genetic information indicates that the HIV-1 has a mutation at codon 55. In certain embodiments, the mutation at codon 55 encodes asparagine (N). In certain embodiments, the genetic information indicates that the HIV-1 has a mutation at codon 66. In certain embodiments, the mutation at codon 66 encodes valine (V) or phenylalanine (F). In certain embodiments, the genetic information indicates that the HIV-1 has a mutation at codon 67. In certain embodiments, the mutation at codon 67 encodes phenylalanine (F). In certain embodiments, the genetic information indicates that the HIV-1 has a mutation at codon 71. In certain embodiments, the mutation at codon 71 encodes leucine (L). In certain embodiments, the genetic information indicates that the HIV-1 has a mutation at codon 72. In certain embodiments, the mutation at codon 72 encodes leucine (L). In certain embodiments, the genetic information indicates that the HIV-1 has a mutation at codon 74. In certain embodiments, the mutation at codon 74 encodes proline (P). In certain embodiments, the genetic information indicates that the HIV-1 has a mutation at codon 79. In certain embodiments, the mutation at codon 79 encodes alanine (A) or serine (S). In certain embodiments, the genetic information indicates that the HIV-1 has a mutation at codon 82. In certain embodiments, the mutation at codon 82 encodes phenylalanine (F), leucine (L) or cysteine (C). In certain embodiments, the genetic information indicates that the HIV-1 has a mutation at codon 91. In certain embodiments, the mutation at codon 91 encodes serine (S). In certain embodiments, the genetic information indicates that the HIV-1 has a mutation at codon 95. In certain embodiments, the mutation at codon 95 encodes phenylalanine (95).

In certain embodiments, the genetic information further indicates that the HIV-1 has a gene encoding protease with a mutation in at least one of codons 4, 10, 12, 13, 15, 16, 18, 19, 20, 22, 23, 24, 34, 35, 36, 37, 43, 46, 48, 53, 55, 57, 58, 60, 61, 62, 63, 66, 67, 69, 71, 72, 74, 79, 82, 83, 85, 90, 91, 92, and 95 in combination with at least one of codons 11, 32, 33, 47, 50, 54, 73, 76, 84 and 89. In certain embodiments, the genetic information indicates a combination of mutations comprising a mutation at codon 11. In certain embodiments, the mutation at codon 11 encodes isoleucine (I). In certain embodiments, the genetic information indicates a combination of mutations comprising a mutation at codon 32. In certain embodiments, the mutation at codon 32 encodes isoleucine (I). In certain embodiments, the genetic information indicates a combination of mutations comprising a mutation at codon 33. In certain embodiments, the mutation at codon 33 encodes phenylalanine (F). In certain embodiments, the genetic information indicates a combination of mutations comprising a mutation at codon 47. In certain embodiments, the mutation at codon 47 encodes valine (V). In certain embodiments, the genetic information indicates a combination of mutations comprising a mutation at codon 50. In certain embodiments, the mutation at codon 50 encodes valine (V). In certain embodiments, the genetic information indicates a combination of mutations comprising a mutation at codon 54. In certain embodiments, the mutation at codon 54 encodes leucine (L), methionine (M), serine (S) or threonine (T). In certain embodiments, the genetic information indicates a combination of mutations comprising a mutation at codon 73. In certain embodiments, the mutation at codon 73 encodes alanine (A), serine (S) or threonine (T). In certain embodiments, the genetic information indicates a combination of mutations comprising a mutation at codon 76. In certain embodiments, the mutation at codon 76 encodes valine (V). In certain embodiments, the genetic information indicates a combination of mutations comprising a mutation at codon 84. In certain embodiments, the mutation at codon 84 encodes valine (V). In certain embodiments, the genetic information indicates a combination of mutations comprising a mutation at codon 89. In certain embodiments, the mutation at codon 89 encodes valine (V).

In certain embodiments, the invention provides a computer-implemented method for determining that an HIV-1 has reduced susceptibility to DRV, comprising inputting genetic information into a memory system of a computer, wherein the genetic information indicates that the HIV-1 has a gene encoding gag with a mutation in at least one of 423, 425, 428, 431, 435, 437, 441, 449, 451, 452, 453, 459, 462, 463, 465, 467, 468, 469, 479, 488 and 497, inputting a correlation between the presence of the mutations and reduced susceptibility to DRV into the memory system of the computer, and determining that the HIV-1 has reduced susceptibility to DRV. In certain embodiments, the genetic information indicates that the HIV-1 has a mutation at codon 431. In certain embodiments, the mutation at codon 431 encodes isoleucine (I). In certain embodiments, the genetic information indicates that the HIV-1 has a mutation at codon 449. In certain embodiments, the mutation at codon 449 encodes valine (V). In certain embodiments, the genetic information indicates that the HIV-1 has a mutation at codon 452. In certain embodiments, the mutation at codon 452 encodes serine (S). In certain embodiments, the genetic information indicates that the HIV-1 has a mutation at codon 453. In certain embodiments, the mutation at codon 453 encodes valine (V) or isoleucine (I). In certain embodiments, the genetic information indicates that the HIV-1 has a mutation at codon 463. In certain embodiments, the mutation at codon 463 glutamic acid (E) or serine (S).

Increased APV and/or DRV Susceptibility

In certain embodiments, the invention provides a computer-implemented method for determining that an HIV-1 has increased susceptibility to DRV, comprising inputting genetic information into a memory system of a computer, wherein the genetic information indicates that the HIV-1 has a gene encoding protease with a mutation in at least one of 30, 43, 45, 50, 63, 64, 71, 77, 88 and 93, inputting a correlation between the presence of the mutations and increased susceptibility to APV into the memory system of the computer, and determining that the HIV-1 has increased susceptibility to DRV. In certain embodiments, the genetic information indicates that the HIV-1 has a mutation at codon 30. In certain embodiments, the mutation at codon 30 encodes asparagine (N). In certain embodiments, the genetic information indicates that the HIV-1 has a mutation at codon 43. In certain embodiments, the mutation at codon 43 encodes arginine (R). In certain embodiments, the genetic information indicates that the HIV-1 has a mutation at codon 45. In certain embodiments, the mutation at codon 45 encodes arginine (R). In certain embodiments, the genetic information indicates that the HIV-1 has a mutation at codon 50. In certain embodiments, the mutation at codon 50 encodes leucine (L). In certain embodiments, the genetic information indicates that the HIV-1 has a mutation at codon 63. In certain embodiments, the mutation at codon 63 encodes serine (S), alanine (A), or glutamine (Q). In certain embodiments, the genetic information indicates that the HIV-1 has a mutation at codon 64. In certain embodiments, the mutation at codon 64 encodes valine (V). In certain embodiments, the genetic information indicates that the HIV-1 has a mutation at codon 71. In certain embodiments, the mutation at codon 71 encodes threonine (T). In certain embodiments, the genetic information indicates that the HIV-1 has a mutation at codon 77. In certain embodiments, the mutation at codon 77 encodes isoleucine (I). In certain embodiments, the genetic information indicates that the HIV-1 has a mutation at codon 88. In certain embodiments, the mutation at codon 88 encodes aspartic acid (D) or serine (S). In certain embodiments, the genetic information indicates that the HIV-1 has a mutation at codon 93. In certain embodiments, the mutation at codon 93 encodes leucine (L).

In certain embodiments, the invention provides a computer-implemented method for determining that an HIV-1 has increased susceptibility to DRV, comprising inputting genetic information into a memory system of a computer, wherein the genetic information indicates that the HIV-1 has a gene encoding gag with a mutation in at least one of 441, 451, 486, 498 and 499, inputting a correlation between the presence of the mutations and increased susceptibility to APV into the memory system of the computer, and determining that the HIV-1 has increased susceptibility to DRV. In certain embodiments, the genetic information indicates that the HIV-1 has a mutation at codon 441. In certain embodiments, the mutation at codon 441 encodes tyrosine (Y). In certain embodiments, the genetic information indicates that the HIV-1 has a mutation at codon 451. In certain embodiments, the mutation at codon 451 encodes asparagines (N). In certain embodiments, the genetic information indicates that the HIV-1 has a mutation at codon 486. In certain embodiments, the mutation at codon 486 encodes serine (S). In certain embodiments, the genetic information indicates that the HIV-1 has a mutation at codon 498. In certain embodiments, the mutation at codon 498 encodes leucine (L). In certain embodiments, the genetic information indicates that the HIV-1 has a mutation at codon 499. In certain embodiments, the mutation at codon 499 encodes leucine (L).

In certain embodiments, the invention provides a computer-implemented method for determining that an HIV-1 has increased susceptibility to DRV, comprising inputting genetic information into a memory system of a computer, wherein the genetic information indicates that the HIV-1 has a gene encoding protease with a mutation in at least one of 20, 30, 36, 41, 43, 45, 50, 63, 64, 65, 70, 71, 74, 75, 77, 82, 88 and 93, inputting a correlation between the presence of the mutations and increased susceptibility to DRV into the memory system of the computer, and determining that the HIV-1 has increased susceptibility to DRV. In certain embodiments, the genetic information indicates that the HIV-1 has a mutation at codon 43. In certain embodiments, the mutation at codon 43 is arginine (R). In certain embodiments, the genetic information indicates that the HIV-1 has a mutation at codon 45. In certain embodiments, the mutation at codon 45 is arginine (R). In certain embodiments, the genetic information indicates that the HIV-1 has a mutation at codon 63. In certain embodiments, the mutation at codon 63 is glutamine (Q).

In certain embodiments, the genetic information further indicates that the HIV-1 has a gene encoding protease with a mutation in at least one of codons 20, 30, 36, 41, 43, 45, 50, 63, 64, 65, 70, 71, 74, 75, 77, 82, 88 and 93 in combination with at least one of codons 30, 50 or 88. In certain embodiments, the genetic information indicates a combination of mutations comprising a mutation at codon 30. In certain embodiments, the mutation at codon 30 encodes asparagine (N). In certain embodiments, the genetic information indicates a combination of mutations comprising a mutation at codon 50. In certain embodiments, the mutation at codon 50 encodes leucine (L). In certain embodiments, the genetic information indicates a combination of mutations comprising a mutation at codon 88. In certain embodiments, the mutation at codon 88 encodes aspartic acid (D) or serine (S).

In certain embodiments, the invention provides a computer-implemented method for determining that an HIV-1 has increased susceptibility to DRV, comprising inputting genetic information into a memory system of a computer, wherein the genetic information indicates that the HIV-1 has a gene encoding gag with a mutation in at least one of codons 437, 439, 441, 442, 451, 475, 480, 482, 483, 486, 498 and 499, inputting a correlation between the presence of the mutations and increased susceptibility to DRV into the memory system of the computer, and determining that the HIV-1 has increased susceptibility to DRV. In certain embodiments, the genetic information indicates that the HIV-1 has a mutation at codon 437. In certain embodiments, the mutation at codon 437 encodes leucine (L). In certain embodiments, the genetic information indicates that the HIV-1 has a mutation at codon 439. In certain embodiments, the mutation at codon 439 encodes serine (S). In certain embodiments, the genetic information indicates that the HIV-1 has a mutation at codon 480. In certain embodiments, the mutation at codon 480 results in deletion of the codon. In certain embodiments, the genetic information indicates that the HIV-1 has a mutation at codon 483. In certain embodiments, the mutation at codon 483 encodes arginine (R). In certain embodiments, the genetic information indicates that the HIV-1 has a mutation at codon 498. In certain embodiments, the mutation at codon 498 encodes leucine (L).

In certain embodiments, the methods further comprise displaying that the HIV-1 has a reduced or increased susceptibility to DRV on a display of the computer. In certain embodiments, the methods further comprise printing that the HIV-1 has a reduced or increased susceptibility to DRV.

In another aspect, the invention provides a print-out indicating that an HIV-1 has a reduced or increased susceptibility to DRV produced according to a method of the invention. In still another aspect, the invention provides a computer-readable medium comprising data indicating that an HIV- has a reduced or increased susceptibility to DRV produced according to a method of the invention.

In yet another aspect, the invention provides a computer-readable medium that comprises data indicating that an HIV-1 has a reduced or increased susceptibility to DRV produced according a method of the invention.

In still another aspect, the invention provides an article of manufacture that comprises computer-readable instructions for performing a method of the invention.

In yet another aspect, the invention provides a computer system that is configured to perform a method of the invention.

### 5.5.4 Viruses and Viral Samples

A mutation associated with altered APV and/or DRV susceptibility according to the present invention can be present in any type of virus. For example, such mutations may be identified in any virus that infects animals known to one of skill in the art without limitation. In one embodiment of the invention, the virus includes viruses known to infect mammals, including dogs, cats, horses, sheep, cows etc. In certain embodiment, the virus is known to infect primates. In preferred embodiments, the virus is known to infect humans. Examples of such viruses that infect humans include, but are not limited to, human immunodeficiency virus (“HIV”), herpes simplex virus, cytomegalovirus virus, varicella zoster virus, other human herpes viruses, influenza A, B and C virus, respiratory syncytial virus, hepatitis A, B and C viruses, rhinovirus, and human papilloma virus. In certain embodiments, the virus is HCV. In other embodiments, the virus is HBV. In a preferred embodiment of the invention, the virus is HIV. Even more preferably, the virus is human immunodeficiency virus type 1 (“HIV-1”). The foregoing are representative of certain viruses for which there is presently available anti viral chemotherapy and represent the viral families retroviridae, herpesviridae, orthomyxoviridae, paramxyxoviridae, picornaviridae, flaviviridae, pneumoviridae and hepadnaviridae. This invention can be used with other viral infections due to other viruses within these families as well as viral infections arising from viruses in other viral families for which there is or there is not a currently available therapy.

A mutation associated with altered APV and/or DRV susceptibility according to the present invention can be found in a viral sample obtained by any means known in the art for obtaining viral samples. Such methods include, but are not limited to, obtaining a viral sample from a human or an animal infected with the virus or obtaining a viral sample from a viral culture. In one embodiment, the viral sample is obtained from a human individual infected with the virus. The viral sample could be obtained from any part of the infected individual's body or any secretion expected to contain the virus. Examples of such parts include, but are not limited to blood, serum, plasma, sputum, lymphatic fluid, semen, vaginal mucus and samples of other bodily fluids. In a preferred embodiment, the sample is a blood, serum or plasma sample.

In another embodiment, a mutation associated with altered APV and/or DRV susceptibility according to the present invention is present in a virus that can be obtained from a culture. In some embodiments, the culture can be obtained from a laboratory. In other embodiments, the culture can be obtained from a collection, for example, the American Type Culture Collection.

In certain embodiments, a mutation associated with altered APV and/or DRV susceptibility according to the present invention is present in a derivative of a virus. In one embodiment, the derivative of the virus is not itself pathogenic. In another embodiment, the derivative of the virus is a plasmid-based system, wherein replication of the plasmid or of a cell transfected with the plasmid is affected by the presence or absence of the selective pressure, such that mutations are selected that increase resistance to the selective pressure. In some embodiments, the derivative of the virus comprises the nucleic acids or proteins of interest, for example, those nucleic acids or proteins to be targeted by an anti-viral treatment. In one embodiment, the genes of interest can be incorporated into a vector. See, e.g., U.S. Pat. Nos. 5,837,464 and 6,242,187 and PCT publication, WO 99/67427, each of which is incorporated herein by reference. In certain embodiments, the genes can be those that encode for a protease or reverse transcriptase.

In another embodiment, the intact virus need not be used. Instead, a part of the virus incorporated into a vector can be used. Preferably that part of the virus is used that is targeted by an anti-viral drug.

In another embodiment, a mutation associated with altered APV and/or DRV susceptibility according to the present invention is present in a genetically modified virus. The virus can be genetically modified using any method known in the art for genetically modifying a virus. For example, the virus can be grown for a desired number of generations in a laboratory culture. In one embodiment, no selective pressure is applied (i.e., the virus is not subjected to a treatment that favors the replication of viruses with certain characteristics), and new mutations accumulate through random genetic drift. In another embodiment, a selective pressure is applied to the virus as it is grown in culture (i.e., the virus is grown under conditions that favor the replication of viruses having one or more characteristics). In one embodiment, the selective pressure is an anti-viral treatment. Any known anti-viral treatment can be used as the selective pressure.

In certain embodiments, the virus is HIV and the selective pressure is a NNRTI. In another embodiment, the virus is HIV-1 and the selective pressure is a NNRTI. Any NNRTI can be used to apply the selective pressure. Examples of NNRTIs include, but are not limited to, nevirapine, delavirdine and efavirenz. By treating HIV cultured in vitro with a NNRTI, one can select for mutant strains of HIV that have an increased resistance to the NNRTI. The stringency of the selective pressure can be manipulated to increase or decrease the survival of viruses not having the selected-for characteristic.

In other embodiments, the virus is HIV and the selective pressure is a NRTI. In another embodiment, the virus is HIV-1 and the selective pressure is a NRTI. Any NRTI can be used to apply the selective pressure. Examples of NRTIs include, but are not limited to, AZT, ddI, ddC, d4T, 3TC, abacavir, and tenofovir. By treating HIV cultured in vitro with a NRTI, one can select for mutant strains of HIV that have an increased resistance to the NRTI. The stringency of the selective pressure can be manipulated to increase or decrease the survival of viruses not having the selected-for characteristic.

In still other embodiments, the virus is HIV and the selective pressure is a PI. In another embodiment, the virus is HIV-1 and the selective pressure is a PI. Any PI can be used to apply the selective pressure. Examples of PIs include, but are not limited to, saquinavir, ritonavir, indinavir, nelfinavir, lopinavir, atazanavir, tipranavir, amprenavir and darunavir. By treating HIV cultured in vitro with a PI, one can select for mutant strains of HIV that have an increased resistance to the PI. The stringency of the selective pressure can be manipulated to increase or decrease the survival of viruses not having the selected-for characteristic.

In still other embodiments, the virus is HIV and the selective pressure is an entry inhibitor. In another embodiment, the virus is HIV-1 and the selective pressure is an entry inhibitor. Any entry inhibitor can be used to apply the selective pressure. An example of a entry inhibitor includes, but is not limited to, fusion inhibitors such as, for example, enfuvirtide. Other entry inhibitors include co-receptor inhibitors, such as, for example, AMD3100 (Anormed). Such co-receptor inhibitors can include any compound that interferes with an interaction between HIV and a co-receptor, e.g., CCR5 or CRCX4, without limitation. By treating HIV cultured in vitro with an entry inhibitor, one can select for mutant strains of HIV that have an increased resistance to the entry inhibitor. The stringency of the selective pressure can be manipulated to increase or decrease the survival of viruses not having the selected-for characteristic.

In another aspect, a mutation associated with altered APV and/or DRV susceptibility according to the present invention can be made by mutagenizing a virus, a viral genome, or a part of a viral genome. Any method of mutagenesis known in the art can be used for this purpose. In certain embodiments, the mutagenesis is essentially random. In certain embodiments, the essentially random mutagenesis is performed by exposing the virus, viral genome or part of the viral genome to a mutagenic treatment. In another embodiment, a gene that encodes a viral protein that is the target of an anti-viral therapy is mutagenized. Examples of essentially random mutagenic treatments include, for example, exposure to mutagenic substances (e.g., ethidium bromide, ethylmethanesulphonate, ethyl nitroso urea (ENU) etc.) radiation (e.g., ultraviolet light), the insertion and/or removal of transposable elements (e.g., Tn5, Tn10), or replication in a cell, cell extract, or in vitro replication system that has an increased rate of mutagenesis. See, e.g., Russell et al., 1979, Proc. Nat. Acad. Sci. USA 76:5918 5922; Russell, W., 1982, Environmental Mutagens and Carcinogens: Proceedings of the Third International Conference on Environmental Mutagens. One of skill in the art will appreciate that while each of these methods of mutagenesis is essentially random, at a molecular level, each has its own preferred targets.

In another aspect, a mutation associated with altered APV and/or DRV susceptibility can be made using site-directed mutagenesis. Any method of site-directed mutagenesis known in the art can be used (see e.g., Sambrook et al., 2001, Molecular Cloning: A Laboratory Manual, Cold Spring Harbor Laboratory, 3rd ed., NY; and Ausubel et al., 1989, Current Protocols in Molecular Biology, Greene Publishing Associates and Wiley Interscience, NY). See, e.g., Sarkar and Sommer, 1990, Biotechniques, 8:404-407. The site directed mutagenesis can be directed to, e.g., a particular gene or genomic region, a particular part of a gene or genomic region, or one or a few particular nucleotides within a gene or genomic region. In one embodiment, the site directed mutagenesis is directed to a viral genomic region, gene, gene fragment, or nucleotide based on one or more criteria. In one embodiment, a gene or a portion of a gene is subjected to site-directed mutagenesis because it encodes a protein that is known or suspected to be a target of an anti-viral therapy, e.g., the gene encoding the HIV reverse transcriptase. In another embodiment, a portion of a gene, or one or a few nucleotides within a gene, are selected for site-directed mutagenesis. In one embodiment, the nucleotides to be mutagenized encode amino acid residues that are known or suspected to interact with an anti-viral compound. In another embodiment, the nucleotides to be mutagenized encode amino acid residues that are known or suspected to be mutated in viral strains that are resistant or susceptible or hypersusceptible to one or more antiviral agents. In another embodiment, the mutagenized nucleotides encode amino acid residues that are adjacent to or near in the primary sequence of the protein residues known or suspected to interact with an anti-viral compound or known or suspected to be mutated in viral strains that are resistant or susceptible or hypersusceptible to one or more antiviral agents. In another embodiment, the mutagenized nucleotides encode amino acid residues that are adjacent to or near to in the secondary, tertiary or quaternary structure of the protein residues known or suspected to interact with an anti-viral compound or known or suspected to be mutated in viral strains having an altered replication capacity. In another embodiment, the mutagenized nucleotides encode amino acid residues in or near the active site of a protein that is known or suspected to bind to an anti-viral compound.

### 6. EXAMPLES

### 6.1 Example 1

### Measuring Altered PI Susceptibility Using Resistance Test Vectors

This example provides methods and compositions for accurately and reproducibly measuring the resistance or sensitivity of HIV-1 to antiretroviral drugs including, for example, PIs such as APV and/or DRV.

Patient derived segment(s) corresponding to the HIV protease and reverse transcriptase coding regions were amplified by the reverse transcription polymerase chain reaction method (RT PCR) using viral RNA isolated from viral particles present in the plasma or serum of HIV infected individuals as follows. Viral RNA was isolated from the plasma or serum using oligo-dT magnetic beads (Dynal Biotech, Oslo, Norway), followed by washing and elution of viral RNA. The RT PCR protocol was divided into two steps. A retroviral reverse transcriptase (e.g. Moloney MuLV reverse transcriptase (Roche Molecular Systems, Inc., Branchburg, N.J.; Invitrogen, Carlsbad, Calif.), or avian myeloblastosis virus (AMV) reverse transcriptase, (Boehringer Mannheim, Indianapolis, Ind.) was used to copy viral RNA into cDNA. The cDNA was then amplified using a thermostable DNA polymerase (e.g. Taq (Roche Molecular Systems, Inc., Branchburg, N.J.), Tth (Roche Molecular Systems, Inc., Branchburg, N.J.), PRIMEZYME™ (isolated from Thermus brockianus, Biometra, Gottingen, Germany)) or a combination of thermostable polymerases as described for the performance of “long PCR” (Barnes, W.M., 1994, Proc. Natl. Acad. Sci, USA 91, 2216 20) (e.g. Expand High Fidelity PCR System (Taq+Pwo), (Boehringer Mannheim. Indianapolis, Ind.); GENEAMP XL™ PCR kit (Tth+Vent), (Roche Molecular Systems, Inc., Branchburg, N.J.); or ADVANTAGE II®, Clontech, Palo Alto, Calif.)

PCR primers were designed to introduce ApaI and PinAI recognition sites into the 5′ or 3′ end of the PCR product, respectively.

Resistance test vectors incorporating the “test” patient derived segments were constructed as described in U.S. Pat. No. 5,837,464 using an amplified DNA product of 1.5 kB prepared by RT PCR using viral RNA as a template and sets of oligonucleotides as primers, followed by digestion with ApaI and AgeI or the isoschizomer PinA1. To ensure that the plasmid DNA corresponding to the resultant fitness test vector comprises a representative sample of the HIV viral quasi-species present in the serum of a given patient, many (>250) independent E. coli transformants obtained in the construction of a given fitness test vector are pooled and used for the preparation of plasmid DNA.

A packaging expression vector encoding an amphotrophic MuLV 4070A env gene product enables production in a resistance test vector host cell of resistance test vector viral particles which can efficiently infect human target cells. Resistance test vectors encoding all HIV genes, with the exception of env, and containing a functional luciferase cassette were used to transfect a packaging host cell (once transfected the host cell is referred to as a fitness test vector host cell). The packaging expression vector which encodes the amphotrophic MuLV 4070A env gene product is used with the resistance test vector to enable production in the resistance test vector host cell of infectious pseudotyped resistance test vector viral particles.

Drug resistance tests performed with resistance test vectors were carried out using packaging host and target host cells consisting of the human embryonic kidney cell line 293.

Resistance tests were carried out with resistance test vectors using two host cell types. Resistance test vector viral particles were produced by a first host cell (the resistance test vector host cell) that was prepared by transfecting a packaging host cell with the resistance test vector and the packaging expression vector. The resistance test vector viral particles were then used to infect a second host cell (the target host cell) in which the expression of the indicator gene is measured.

The amount of luciferase activity detected in infected cells is used as a direct measure of “infectivity,” i.e., the ability of the virus to complete a single round of replication. Thus, drug resistance or sensitivity can be determined by plotting the amount of luciferase activity produced by patient derived viruses in the presence of varying concentrations of the antiviral drug. By identifying the concentration of drug at which luciferase activity is half-maximum, the IC50 of the virus from which patient-derived segment(s) were obtained for the antiretroviral agent can be determined.

Host (293) cells were seeded in 10 cm diameter dishes and were transfected one day after plating with resistance test vector plasmid DNA and the envelope expression vector. Transfections were performed using a calcium phosphate co-precipitation procedure. The cell culture media containing the DNA precipitate was replaced with fresh medium, from one to 24 hours, after transfection. Cell culture medium containing resistance test vector viral particles was harvested one to four days after transfection and was passed through a 0.45 mm filter before optional storage at 80° C. Before infection, target cells (293 cells) were plated in cell culture media. Control infections were performed using cell culture media from mock transfections (no DNA) or transfections containing the resistance test vector plasmid DNA without the envelope expression plasmid. One to three or more days after infection the media was removed and cell lysis buffer (Promega Corp.; Madison, Wis.) was added to each well. Cell lysates were assayed for luciferase activity. Alternatively, cells were lysed and luciferase was measured by adding Steady Glo (Promega Corp.; Madison, Wis.) reagent directly to each well without aspirating the culture media from the well. The amount of luciferase activity produced in infected cells was normalized to adjust for variation in transfection efficiency in the transfected host cells by measuring the luciferase activity in the transfected cells, which is not dependent on viral gene functions, and adjusting the luciferase activity from infected cell accordingly.

### 6.2 Example 2

### Identifying Mutations Correlated with Reduced Susceptibility to APV and/or DRV

This example provides methods and compositions for identifying mutations that correlate with altered susceptibility to APV or DRV. Resistance test vectors were constructed and used as described in Example 1. Resistance test vectors derived from patient samples or clones derived from the resistance test vector pools were tested in a resistance assay to determine accurately and quantitatively the relative APV or DRV resistance or susceptibility compared to the median observed resistance or susceptibility.

Genotypic Analysis of Patient HIV Samples:

Resistance test vector DNAs, either pools or clones, can be analyzed by any genotyping method, e.g., as described above. In this example, patient HIV sample sequences were determined using viral RNA purification, RT/PCR and ABI chain terminator automated sequencing. The sequence that was determined was compared to that of a reference sequence, NL4-3. The genotype was examined for sequences that were different from the reference or pre-treatment sequence and correlated to the observed IC₅₀ for APV and DRV.

Correlation of Mutations with Altered Susceptibility to APV and/or DRV:

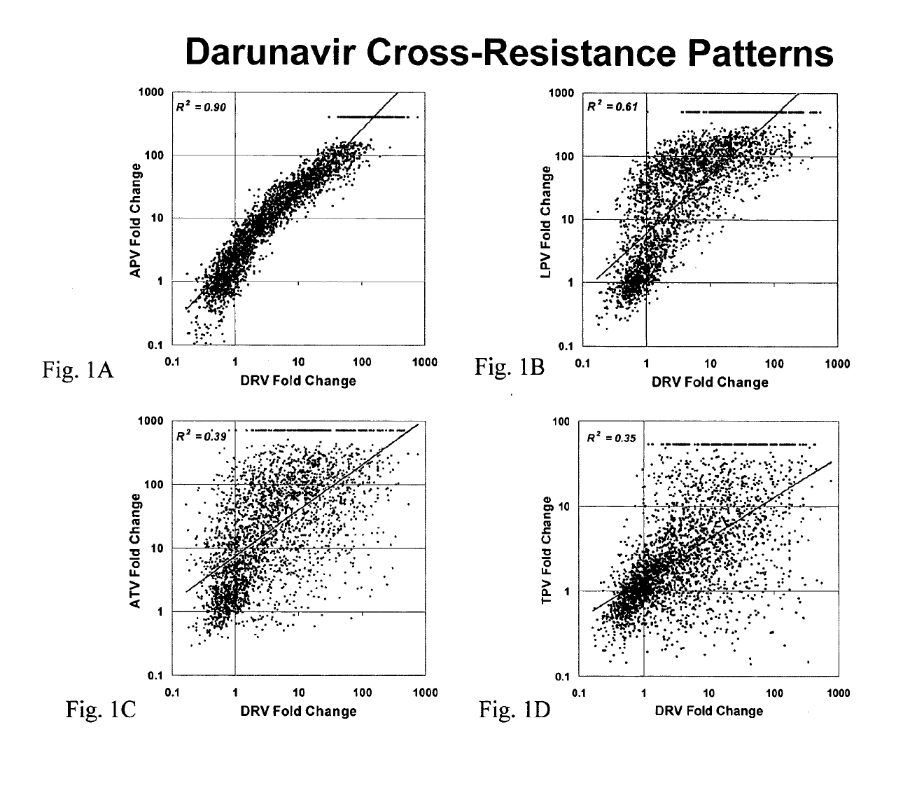

To identify mutations associated with altered PI susceptibility, the following analysis was performed. A collection of 2862 samples that comprise at least one major PI resistance-associated mutation at positions L23, L24, D30, V32, M46, I47, G48, I50, I54, V82 (except V82I), 184, N88, and L90, with no mixtures at V11I, V32I, L33F, 147V, I50V, I54L or M, G73S, L76V, 184V or L89V, in the HIV-1 protease. were identified and their DRV, APV, LPV, ATV, and TPV susceptibility phenotypes were determined as described above. Viruses that exhibit an IC₅₀ 2-fold higher than wild-type virus were designated as having reduced susceptibility to the respective PI. Viruses that exhibit an IC₅₀ 0.4 fold lower than wild-type virus were designated as having increased susceptibility to the respective PI. Isolates without NRTI, NNRTI or PI mutations served as a wild type (WT) reference group. Scatter plots (See FIG. 1) were generated using Prism 4.0 (GraphPad, San Diego, Calif.).

Mutations Associated with Altered APV or DRV Susceptibility

The experiments described above identified a number of mutations that significantly correlate with reduced or increased susceptibility to APV and/or DRV. Table 1 presents P-values and odds ratios corresponding to mutations associated with altered susceptibility (FC>2 or <0.4) to APV or DRV. Mutations in gag (amino acids 418-500) or protease associated with reduced susceptibility (fold change>2) were determined using Fisher's Exact test with correction for multiple comparisons. Odds ratios were determined by dividing the percentage of virus sampled which display altered APV or DRV susceptibility (FC>2) and comprise the mutation by the percentage of virus which did not display altered APV or DRV susceptibility and comprise the mutation. Mutations with an odds ratio>1 correlate with reduced susceptibility. Mutations with an odds ratio<1 negatively correlate with reduced susceptibility. Mutations with an odds ratio having the designation “>>>” as shown in Table 1 were observed only in viruses exhibiting a reduced susceptibility phenotype (FC>2), i.e. were never observed in a virus displaying a non-altered susceptibility phenotype.

### 6.3 Example 3

### Correlation of APV and DRV Susceptibility

This example demonstrates the correlation between mutations which associate with APV susceptibility and DRV susceptibility. Phenotypic assays for the PIs DRV, APV, LPV, ATV, and TPV susceptibility were performed on a collection of 2862 samples as described above. The FC in susceptibility relative to wild-type (NL4-3) for each of APV, LPV, ATV and TPV respectively were then plotted as a histogram against the FC in susceptibility for DRV. As shown in FIG. 1, APV FC tightly correlated with DRV FC, while no correlation was observed between DRV and LPV, ATV and TPV respectively.

Further, a significant overlap was observed for mutations having significant P-values and odds ratios for both APV and DRV. Table 2 presents mutations having significant P-values for either APV or DRV that are associated with reduced susceptibility. Of the 60 mutations most strongly correlated with reduced susceptibility to APV (OR>3), 44 are also strongly correlated with reduced susceptibility to DRV (OR>3). Table 3 presents mutations having significant P-values for either APV or DRV that are associated with increased susceptibility. Of the 9 mutations most strongly correlated with increased susceptibility to APV (OR<0.5), 8 are also strongly correlated with increased susceptibility to DRV (OR<0.5). In both cases, odds ratios for APV show a strong correlation with the odds ratios for DRV. In sum, these data show that altered susceptibility to APV significantly correlates with altered susceptibility to DRV.

### 7. Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

TABLE 1
Mutations Associated with Altered Susceptibility to APV or DRV

| | | | | AMP | | DRV | |
|---|---|---|---|---|---|---|---|
| | | PR or | | | Odds | | Odds |
| | Mutation | gag | n Mut | P-value | Ratio | P-value | Ratio |
| 1 | V11L | PR | 56 | 1.6E−06 | >>> | 4.3E−13 | >>> |
| 2 | I50V | PR | 104 | 2.7E−12 | >>> | 1.0E−24 | >>> |
| 3 | L89V | PR | 185 | 4.1E−22 | >>> | 1.8E−39 | 44.56 |
| 4 | V32I | PR | 396 | 3.8E−46 | 62.34 | 6.6E−77 | 18.66 |
| 5 | A71L | PR | 47 | 1.6E−05 | >>> | 2.9E−08 | 16.53 |
| 6 | I54M | PR | 207 | 1.1E−21 | 32.44 | 2.4E−35 | 14.47 |
| 7 | I47V | PR | 313 | 7.6E−37 | 98.74 | 6.7E−54 | 13.64 |
| 8 | V11I | PR | 148 | 1.6E−14 | 23.10 | 6.5E−24 | 12.85 |
| 9 | L76V | PR | 146 | 2.1E−17 | >>> | 1.9E−22 | 11.18 |
| 10 | T4A | PR | 31 | 1.3E−02 | 9.49 | 5.5E−05 | 10.65 |
| 11 | K55N | PR | 30 | 1.4E−03 | >>> | 9.0E−05 | 10.28 |
| 12 | G73A | PR | 39 | 1.2E−02 | 5.85 | 1.1E−05 | 8.81 |
| 13 | L24F | PR | 38 | 2.9E−03 | 11.71 | 1.7E−05 | 8.57 |
| 14 | G73T | PR | 140 | 1.0E−13 | 21.84 | 2.6E−18 | 7.84 |
| 15 | L19P | PR | 43 | 3.8E−03 | 6.49 | 1.4E−05 | 7.16 |
| 16 | I84V | PR | 923 | 1.6E−124 | 58.10 | 2.5E−148 | 7.06 |
| 17 | P453V | gag | 31 | 1.3E−02 | 9.49 | 5.6E−04 | 6.86 |
| 18 | L33F | PR | 900 | 7.2E−93 | 12.07 | 6.2E−140 | 6.78 |
| 19 | T74P | PR | 178 | 4.4E−18 | 27.85 | 4.8E−20 | 6.15 |
| 20 | P453I | gag | 56 | 4.9E−03 | 4.11 | 1.8E−06 | 6.12 |
| 21 | A431I | gag | 69 | 1.1E−06 | 21.52 | 2.3E−07 | 5.60 |
| 22 | I54L | PR | 229 | 2.2E−17 | 8.74 | 4.5E−24 | 5.50 |
| 23 | V82F | PR | 76 | 2.2E−06 | 11.71 | 6.5E−08 | 5.47 |
| 24 | V82L | PR | 42 | 5.5E−03 | 6.33 | 1.1E−04 | 5.44 |
| 25 | P79A | PR | 58 | 1.6E−05 | 18.04 | 3.4E−06 | 5.35 |
| 26 | P79S | PR | 31 | 1.3E−02 | 9.49 | 2.4E−03 | 4.96 |
| 27 | I66V | PR | 99 | 5.8E−05 | 4.16 | 2.1E−09 | 4.86 |
| 28 | L449V | gag | 186 | 1.5E−14 | 9.49 | 2.2E−17 | 4.73 |
| 29 | T91S | PR | 145 | 7.3E−12 | 11.16 | 1.8E−12 | 4.34 |
| 30 | A22V | PR | 45 | 1.3E−02 | 4.43 | 6.9E−04 | 3.99 |
| 31 | R452S | gag | 171 | 1.1E−05 | 2.87 | 2.9E−13 | 3.92 |
| 32 | G16A | PR | 152 | 8.9E−08 | 4.49 | 4.1E−11 | 3.73 |
| 33 | C67F | PR | 91 | 4.2E−07 | 9.28 | 7.4E−07 | 3.72 |
| 34 | K43T | PR | 406 | 2.6E−29 | 7.24 | 2.6E−31 | 3.72 |
| 35 | V82C | PR | 60 | 9.9E−05 | 9.18 | 1.2E−04 | 3.67 |
| 36 | I54S | PR | 41 | 9.9E−05 | >>> | 2.4E−03 | 3.57 |
| 37 | F53Y | PR | 34 | 6.0E−03 | 10.44 | 7.7E−03 | 3.43 |
| 38 | F463E | gag | 34 | 2.0E−01 | 2.37 | 7.7E−03 | 3.43 |
| 39 | K20V | PR | 90 | 1.1E−04 | 4.43 | 3.6E−06 | 3.40 |
| 40 | E34Q | PR | 184 | 1.4E−09 | 4.54 | 4.5E−12 | 3.36 |
| 41 | I66F | PR | 114 | 1.0E−05 | 4.19 | 3.5E−07 | 3.25 |
| 42 | F463S | gag | 48 | 1.7E−03 | 7.28 | 2.1E−03 | 3.18 |
| 43 | C95F | PR | 100 | 1.1E−05 | 4.96 | 4.3E−06 | 3.13 |
| 44 | T4P | PR | 41 | 2.4E−02 | 4.01 | 6.8E−03 | 3.03 |
| 45 | I54T | PR | 41 | 9.9E−05 | >>> | 6.8E−03 | 3.03 |
| 46 | I72L | PR | 133 | 1.2E−05 | 3.51 | 1.0E−07 | 3.02 |
| 47 | R452K | gag | 71 | 2.8E−02 | 2.49 | 3.0E−04 | 2.99 |
| 48 | E35N | PR | 99 | 7.8E−08 | 10.13 | 1.8E−05 | 2.90 |
| 49 | F463V | gag | 74 | 3.2E−02 | 2.29 | 2.7E−04 | 2.89 |
| 50 | Q92K | PR | 179 | 8.2E−13 | 7.78 | 2.7E−09 | 2.82 |
| 51 | L24M | PR | 29 | 6.6E−02 | 4.27 | 4.8E−02 | 2.82 |
| 52 | T4S | PR | 53 | 5.1E−04 | 8.07 | 3.4E−03 | 2.80 |
| 53 | I85V | PR | 290 | 6.6E−11 | 3.21 | 1.3E−14 | 2.76 |
| 54 | K55R | PR | 496 | 2.6E−25 | 4.17 | 2.7E−25 | 2.70 |
| 55 | H69R | PR | 95 | 1.2E−04 | 3.98 | 1.4E−04 | 2.59 |
| 56 | L10F | PR | 615 | 3.4E−26 | 3.36 | 1.2E−30 | 2.59 |
| 57 | F463C | gag | 36 | 5.7E−01 | 1.58 | 3.8E−02 | 2.57 |
| 58 | Q61N | PR | 70 | 3.9E−04 | 5.22 | 2.7E−03 | 2.48 |
| 59 | F465S | gag | 165 | 1.3E−03 | 2.17 | 8.6E−07 | 2.45 |
| 60 | G48M | PR | 30 | 1.3E−02 | 9.18 | 8.0E−02 | 2.41 |
| 61 | Q58E | PR | 471 | 1.6E−25 | 4.49 | 1.5E−19 | 2.41 |
| 62 | E428D | gag | 107 | 2.9E−02 | 1.94 | 1.5E−04 | 2.41 |
| 63 | E467K | gag | 77 | 2.5E−02 | 2.39 | 2.1E−03 | 2.41 |
| 64 | T91A | PR | 34 | 9.0E−02 | 3.27 | 7.1E−02 | 2.39 |
| 65 | I54A | PR | 76 | 1.8E−05 | 7.70 | 2.8E−03 | 2.37 |
| 66 | V82I | PR | 116 | 5.8E−03 | 2.31 | 1.9E−04 | 2.31 |
| 67 | F465M | gag | 29 | 6.6E−02 | 4.27 | 1.1E−01 | 2.31 |
| 68 | F53L | PR | 314 | 1.5E−17 | 4.91 | 1.9E−11 | 2.30 |
| 69 | S462N | gag | 103 | 1.3E−02 | 2.19 | 5.6E−04 | 2.29 |
| 70 | E34D | PR | 49 | 2.3E−01 | 1.90 | 3.0E−02 | 2.26 |
| 71 | G73C | PR | 73 | 6.7E−03 | 2.98 | 5.1E−03 | 2.24 |
| 72 | E468K | gag | 73 | 6.7E−03 | 2.98 | 5.1E−03 | 2.24 |
| 73 | M36L | PR | 196 | 1.4E−03 | 1.98 | 1.1E−06 | 2.20 |
| 74 | L89I | PR | 67 | 2.3E−02 | 2.71 | 1.1E−02 | 2.16 |
| 75 | L23I | PR | 81 | 1.4E−04 | 4.81 | 7.7E−03 | 2.10 |
| 76 | N83D | PR | 50 | 2.3E−01 | 1.94 | 4.6E−02 | 2.09 |
| 77 | E419D | gag | 42 | 7.2E−01 | 1.34 | 8.0E−02 | 2.07 |
| 78 | Q18H | PR | 76 | 6.6E−02 | 2.09 | 1.2E−02 | 2.06 |
| 79 | M423I | gag | 136 | 6.2E−03 | 2.07 | 4.5E−04 | 2.04 |
| 80 | G435E | gag | 68 | 1.6E−01 | 1.84 | 2.2E−02 | 2.04 |
| 81 | S462del | gag | 34 | 2.0E−01 | 2.37 | 1.4E−01 | 2.04 |
| 82 | K70T | PR | 45 | 5.3E−01 | 1.46 | 6.9E−02 | 2.02 |
| 83 | S488A | gag | 206 | 4.5E−03 | 1.79 | 8.8E−06 | 2.02 |
| 84 | Q92R | PR | 56 | 2.0E−01 | 1.90 | 4.5E−02 | 2.01 |
| 85 | G435R | gag | 41 | 5.3E−01 | 1.54 | 1.0E−01 | 2.00 |
| 86 | H441Q | gag | 245 | 1.3E−08 | 3.05 | 1.3E−06 | 1.99 |
| 87 | I437V | gag | 435 | 1.3E−18 | 3.51 | 2.4E−11 | 1.95 |
| 88 | T469K | gag | 113 | 5.8E−02 | 1.79 | 2.9E−03 | 1.94 |
| 89 | A431V | gag | 1181 | 2.2E−59 | 3.21 | 1.0E−40 | 1.94 |
| 90 | K418N | gag | 40 | 8.6E−02 | 2.85 | 1.3E−01 | 1.94 |
| 91 | M46I | PR | 1283 | 2.1E−42 | 2.32 | 1.9E−45 | 1.92 |
| 92 | P453A | gag | 83 | 8.3E−02 | 1.87 | 1.7E−02 | 1.92 |
| 93 | G73S | PR | 389 | 7.2E−12 | 2.69 | 1.8E−09 | 1.91 |
| 94 | E461del | gag | 36 | 1.5E−01 | 2.53 | 1.5E−01 | 1.91 |
| 95 | K20R | PR | 731 | 1.6E−20 | 2.41 | 2.2E−19 | 1.90 |
| 96 | F465C | gag | 81 | 1.0E−01 | 1.82 | 2.8E−02 | 1.85 |
| 97 | T12P | PR | 119 | 3.4E−03 | 2.37 | 5.1E−03 | 1.84 |
| 98 | V82T | PR | 224 | 5.7E−08 | 3.06 | 5.3E−05 | 1.84 |
| 99 | A457V | gag | 42 | 1.4E−01 | 2.34 | 1.4E−01 | 1.84 |
| 100 | P453L | gag | 919 | 7.6E−10 | 1.58 | 1.1E−22 | 1.80 |
| 101 | K70E | PR | 48 | 2.8E−01 | 1.85 | 1.3E−01 | 1.78 |
| 102 | S451T | gag | 123 | 3.2E−02 | 1.85 | 7.7E−03 | 1.78 |
| 103 | N37E | PR | 184 | 1.0E−01 | 1.45 | 7.3E−04 | 1.77 |
| 104 | I13M | PR | 34 | 2.3E−02 | 5.06 | 2.5E−01 | 1.76 |
| 105 | A71I | PR | 256 | 2.0E−04 | 1.93 | 6.8E−05 | 1.74 |
| 106 | I13V | PR | 1282 | 7.7E−10 | 1.41 | 1.2E−31 | 1.71 |
| 107 | Q476K | gag | 76 | 6.6E−02 | 2.09 | 7.0E−02 | 1.69 |
| 108 | F465I | gag | 33 | 6.8E−01 | 1.42 | 3.2E−01 | 1.69 |
| 109 | P459T | gag | 99 | 5.7E−03 | 2.53 | 3.1E−02 | 1.69 |
| 110 | F463L | gag | 316 | 4.6E−03 | 1.57 | 2.0E−05 | 1.68 |
| 111 | E428G | gag | 115 | 5.3E−01 | 1.27 | 2.2E−02 | 1.68 |
| 112 | K424R | gag | 62 | 2.9E−01 | 1.65 | 1.2E−01 | 1.66 |
| 113 | F465L | gag | 186 | 2.1E−02 | 1.65 | 2.9E−03 | 1.66 |
| 114 | F465V | gag | 90 | 1.2E−01 | 1.72 | 6.3E−02 | 1.63 |
| 115 | K418E | gag | 77 | 1.5E−01 | 1.71 | 9.1E−02 | 1.62 |
| 116 | T470P | gag | 51 | 3.0E−01 | 1.70 | 1.9E−01 | 1.61 |
| 117 | G86R | PR | 35 | 8.0E−01 | 1.27 | 3.4E−01 | 1.60 |
| 118 | M46L | PR | 512 | 4.6E−14 | 2.48 | 1.9E−07 | 1.60 |
| 119 | E467G | gag | 92 | 6.8E−01 | 1.22 | 8.1E−02 | 1.60 |
| 120 | T12K | PR | 57 | 1.0E+00 | 1.07 | 1.7E−01 | 1.59 |
| 121 | L63T | PR | 98 | 3.5E−01 | 1.41 | 6.1E−02 | 1.59 |
| 122 | I72K | PR | 41 | 5.3E−01 | 1.54 | 3.1E−01 | 1.58 |
| 123 | M423L | gag | 44 | 1.0E+00 | 1.08 | 2.6E−01 | 1.57 |
| 124 | K20T | PR | 254 | 1.6E−02 | 1.55 | 2.3E−03 | 1.54 |
| 125 | I15V | PR | 751 | 7.0E−08 | 1.60 | 1.1E−09 | 1.53 |
| 126 | N37T | PR | 80 | 9.6E−01 | 1.09 | 1.4E−01 | 1.53 |
| 127 | L89M | PR | 165 | 4.7E−03 | 1.95 | 2.8E−02 | 1.51 |
| 128 | T427I | gag | 106 | 1.0E+00 | 1.03 | 1.1E−01 | 1.49 |
| 129 | D425E | gag | 451 | 1.9E−04 | 1.59 | 5.6E−05 | 1.49 |
| 130 | G16E | PR | 145 | 5.3E−01 | 1.21 | 5.3E−02 | 1.48 |
| 131 | A71V | PR | 1411 | 1.4E−20 | 1.62 | 6.9E−21 | 1.47 |
| 132 | R452G | gag | 30 | 9.1E−01 | 1.27 | 4.9E−01 | 1.47 |
| 133 | P497Q | gag | 105 | 4.7E−02 | 1.90 | 1.3E−01 | 1.47 |
| 134 | I72M | PR | 90 | 7.5E−01 | 1.18 | 1.4E−01 | 1.47 |
| 135 | I72R | PR | 33 | 9.0E−02 | 3.16 | 5.0E−01 | 1.47 |
| 136 | T74A | PR | 63 | 3.6E−01 | 1.50 | 2.5E−01 | 1.47 |
| 137 | F463I | gag | 63 | 3.6E−01 | 1.50 | 2.5E−01 | 1.47 |
| 138 | R464S | gag | 125 | 1.1E−01 | 1.57 | 9.8E−02 | 1.45 |
| 139 | L19V | PR | 107 | 4.5E−01 | 1.30 | 1.3E−01 | 1.45 |
| 140 | L449F | gag | 269 | 3.2E−02 | 1.46 | 7.8E−03 | 1.44 |
| 141 | R429G | gag | 59 | 9.5E−01 | 1.12 | 3.4E−01 | 1.43 |
| 142 | P472L | gag | 44 | 8.3E−01 | 1.23 | 4.1E−01 | 1.42 |
| 143 | I54V | PR | 1189 | 3.3E−47 | 2.67 | 6.6E−13 | 1.42 |
| 144 | E428Q | gag | 38 | 1.0E+00 | 1.02 | 4.6E−01 | 1.41 |
| 145 | H421P | gag | 35 | 6.6E−02 | 3.38 | 5.2E−01 | 1.41 |
| 146 | G466R | gag | 128 | 8.5E−01 | 1.08 | 1.2E−01 | 1.40 |
| 147 | G48V | PR | 156 | 4.6E−07 | 3.80 | 8.9E−02 | 1.39 |
| 148 | D60E | PR | 485 | 4.9E−04 | 1.51 | 5.8E−04 | 1.39 |
| 149 | M36I | PR | 1447 | 5.2E−15 | 1.48 | 8.0E−15 | 1.37 |
| 150 | L10V | PR | 441 | 1.2E−04 | 1.62 | 3.0E−03 | 1.36 |
| 151 | F465E | gag | 105 | 6.3E−01 | 1.19 | 2.5E−01 | 1.35 |
| 152 | D480N | gag | 59 | 2.2E−01 | 1.76 | 4.8E−01 | 1.33 |
| 153 | Q61H | PR | 73 | 2.1E−02 | 2.57 | 4.0E−01 | 1.33 |
| 154 | E35D | PR | 1165 | 9.3E−05 | 1.27 | 2.7E−08 | 1.32 |
| 155 | N37D | PR | 636 | 9.2E−06 | 1.55 | 6.9E−04 | 1.31 |
| 156 | N37H | PR | 39 | 6.0E−01 | 1.45 | 6.4E−01 | 1.31 |
| 157 | I72T | PR | 267 | 2.9E−01 | 1.22 | 5.8E−02 | 1.31 |
| 158 | R429K | gag | 50 | 5.5E−01 | 1.44 | 5.2E−01 | 1.31 |
| 159 | I479T | gag | 516 | 3.1E−02 | 1.28 | 4.2E−03 | 1.30 |
| 160 | L10I | PR | 1382 | 1.9E−33 | 1.95 | 1.5E−09 | 1.29 |
| 161 | I72V | PR | 433 | 1.2E−03 | 1.51 | 1.6E−02 | 1.29 |
| 162 | K418Q | gag | 295 | 1.1E−01 | 1.32 | 6.1E−02 | 1.29 |
| 163 | E35G | PR | 82 | 5.3E−01 | 1.31 | 4.4E−01 | 1.27 |
| 164 | T12A | PR | 30 | 4.2E−01 | 0.63 | 7.1E−01 | 1.27 |
| 165 | S473L | gag | 60 | 3.5E−01 | 1.58 | 5.6E−01 | 1.27 |
| 166 | A487S | gag | 98 | 8.9E−01 | 1.09 | 4.2E−01 | 1.27 |
| 167 | C67Y | PR | 38 | 1.1E−01 | 2.69 | 6.4E−01 | 1.26 |
| 168 | T427N | gag | 184 | 9.9E−01 | 0.98 | 2.3E−01 | 1.25 |
| 169 | E468G | gag | 192 | 3.5E−01 | 1.24 | 2.1E−01 | 1.25 |
| 170 | L90M | PR | 1681 | 1.8E−07 | 1.24 | 4.9E−11 | 1.25 |
| 171 | K20I | PR | 293 | 1.6E−01 | 1.28 | 1.1E−01 | 1.24 |
| 172 | P459L | gag | 43 | 6.2E−01 | 1.38 | 6.6E−01 | 1.24 |
| 173 | L19Q | PR | 51 | 8.4E−01 | 1.15 | 6.1E−01 | 1.24 |
| 174 | I479D | gag | 59 | 9.5E−01 | 1.12 | 6.3E−01 | 1.24 |
| 175 | L19I | PR | 370 | 8.3E−02 | 1.29 | 7.5E−02 | 1.23 |
| 176 | R464K | gag | 306 | 7.9E−01 | 1.07 | 1.3E−01 | 1.22 |
| 177 | P39S | PR | 61 | 9.5E−01 | 0.97 | 6.4E−01 | 1.21 |
| 178 | T471N | gag | 37 | 9.3E−01 | 1.15 | 7.3E−01 | 1.21 |
| 179 | K442N | gag | 29 | 5.3E−01 | 0.70 | 8.1E−01 | 1.20 |
| 180 | Q500K | gag | 58 | 7.7E−01 | 1.21 | 7.1E−01 | 1.20 |
| 181 | R57K | PR | 603 | 2.2E−01 | 1.15 | 4.6E−02 | 1.19 |
| 182 | T427A | gag | 68 | 1.6E−01 | 1.84 | 6.6E−01 | 1.19 |
| 183 | S473A | gag | 60 | 5.3E−01 | 1.41 | 7.1E−01 | 1.18 |
| 184 | I62V | PR | 1600 | 4.3E−08 | 1.28 | 1.4E−05 | 1.17 |
| 185 | T427P | gag | 57 | 3.3E−01 | 0.69 | 7.1E−01 | 1.17 |
| 186 | L33V | PR | 44 | 8.3E−01 | 1.23 | 7.5E−01 | 1.17 |
| 187 | K14R | PR | 238 | 1.2E−01 | 1.36 | 3.6E−01 | 1.17 |
| 188 | K418R | gag | 357 | 6.9E−01 | 1.08 | 2.2E−01 | 1.17 |
| 189 | P39Q | PR | 36 | 5.7E−01 | 1.58 | 8.3E−01 | 1.15 |
| 190 | L24I | PR | 274 | 2.1E−12 | 3.81 | 4.7E−01 | 1.13 |
| 191 | V82A | PR | 1043 | 3.9E−22 | 1.98 | 4.6E−02 | 1.12 |
| 192 | Y484ins | gag | 108 | 2.4E−01 | 1.48 | 7.3E−01 | 1.11 |
| 193 | S440F | gag | 35 | 1.0E+00 | 1.07 | 9.4E−01 | 1.10 |
| 194 | S451G | gag | 45 | 5.3E−01 | 1.46 | 9.4E−01 | 1.10 |
| 195 | I479K | gag | 280 | 5.3E−01 | 1.14 | 5.3E−01 | 1.10 |
| 196 | M46V | PR | 30 | 6.7E−01 | 0.74 | 9.3E−01 | 1.10 |
| 197 | G443E | gag | 40 | 6.0E−01 | 1.49 | 9.4E−01 | 1.10 |
| 198 | I479M | gag | 95 | 5.5E−01 | 1.27 | 7.8E−01 | 1.10 |
| 199 | K481E | gag | 102 | 4.3E−01 | 0.80 | 7.9E−01 | 1.09 |
| 200 | R464G | gag | 161 | 1.0E+00 | 1.02 | 7.3E−01 | 1.08 |
| 201 | E460A | gag | 629 | 2.4E−01 | 1.14 | 4.0E−01 | 1.08 |
| 202 | L19T | PR | 52 | 1.0E+00 | 1.05 | 9.4E−01 | 1.08 |
| 203 | L63H | PR | 52 | 5.6E−01 | 0.78 | 9.4E−01 | 1.08 |
| 204 | K436R | gag | 361 | 1.1E−01 | 1.27 | 5.9E−01 | 1.08 |
| 205 | L483K | gag | 89 | 8.9E−01 | 1.09 | 8.4E−01 | 1.08 |
| 206 | D480K | gag | 79 | 2.0E−01 | 0.68 | 9.0E−01 | 1.08 |
| 207 | T12E | PR | 32 | 4.5E−01 | 1.71 | 1.0E+00 | 1.07 |
| 208 | E460ins | gag | 738 | 1.7E−01 | 1.14 | 4.0E−01 | 1.07 |
| 209 | L63P | PR | 2392 | 1.5E−05 | 1.10 | 2.2E−04 | 1.07 |
| 210 | S473P | gag | 1151 | 7.5E−01 | 1.03 | 4.0E−01 | 1.05 |
| 211 | P472S | gag | 51 | 1.0E+00 | 1.03 | 9.4E−01 | 1.05 |
| 212 | T471A | gag | 422 | 7.2E−01 | 0.94 | 7.4E−01 | 1.05 |
| 213 | T12N | PR | 29 | 1.0E+00 | 0.99 | 1.0E+00 | 1.04 |
| 214 | P478S | gag | 111 | 7.1E−01 | 0.89 | 9.7E−01 | 1.04 |
| 215 | P478Q | gag | 427 | 7.5E−01 | 1.06 | 8.1E−01 | 1.04 |
| 216 | Q61E | PR | 123 | 1.0E+00 | 1.03 | 9.3E−01 | 1.04 |
| 217 | L449P | gag | 224 | 7.5E−01 | 0.93 | 9.1E−01 | 1.03 |
| 218 | S440A | gag | 36 | 5.7E−01 | 1.58 | 1.0E+00 | 1.03 |
| 219 | E467del | gag | 120 | 1.0E+00 | 0.99 | 9.7E−01 | 1.03 |
| 220 | P478A | gag | 36 | 7.0E−01 | 0.82 | 1.0E+00 | 1.03 |
| 221 | K481R | gag | 60 | 1.0E+00 | 1.04 | 1.0E+00 | 1.03 |
| 222 | R490K | gag | 1484 | 9.6E−01 | 1.01 | 6.3E−01 | 1.03 |
| 223 | T470A | gag | 725 | 5.3E−01 | 1.07 | 9.1E−01 | 1.02 |
| 224 | L33I | PR | 143 | 3.5E−01 | 1.30 | 1.0E+00 | 1.02 |
| 225 | C426ins | gag | 100 | 8.3E−01 | 1.12 | 1.0E+00 | 1.01 |
| 226 | I479R | gag | 259 | 8.6E−01 | 1.05 | 9.9E−01 | 1.01 |
| 227 | S495N | gag | 2669 | 6.8E−01 | 1.01 | 5.0E−01 | 1.01 |
| 228 | P478T | gag | 308 | 1.0E+00 | 1.02 | 1.0E+00 | 1.01 |
| 229 | P497L | gag | 33 | 5.6E−01 | 0.73 | 1.0E+00 | 1.00 |
| 230 | G466del | gag | 27 | 9.8E−01 | 0.98 | 1.0E+00 | 0.99 |
| 231 | D480E | gag | 94 | 7.5E−01 | 1.17 | 1.0E+00 | 0.99 |
| 232 | E477G | gag | 152 | 6.4E−01 | 0.89 | 9.7E−01 | 0.98 |
| 233 | H441R | gag | 35 | 9.2E−01 | 0.91 | 1.0E+00 | 0.98 |
| 234 | I479A | gag | 35 | 6.9E−01 | 0.79 | 1.0E+00 | 0.98 |
| 235 | P478K | gag | 86 | 7.4E−01 | 1.20 | 9.6E−01 | 0.97 |
| 236 | I479V | gag | 141 | 2.7E−01 | 0.77 | 9.4E−01 | 0.96 |
| 237 | A487T | gag | 862 | 2.8E−01 | 0.91 | 6.2E−01 | 0.96 |
| 238 | L63C | PR | 39 | 1.0E+00 | 1.05 | 9.4E−01 | 0.95 |
| 239 | T469A | gag | 211 | 9.4E−01 | 0.97 | 8.1E−01 | 0.95 |
| 240 | Q476P | gag | 71 | 1.0E+00 | 1.01 | 9.0E−01 | 0.95 |
| 241 | T471I | gag | 64 | 3.0E−01 | 0.70 | 9.5E−01 | 0.94 |
| 242 | E482G | gag | 107 | 2.8E−01 | 0.74 | 8.5E−01 | 0.94 |
| 243 | K481Q | gag | 102 | 4.3E−01 | 0.80 | 8.5E−01 | 0.93 |
| 244 | T427S | gag | 139 | 5.6E−01 | 1.20 | 7.1E−01 | 0.91 |
| 245 | T456S | gag | 576 | 7.2E−01 | 1.05 | 3.3E−01 | 0.91 |
| 246 | P459S | gag | 44 | 1.0E+00 | 1.08 | 8.5E−01 | 0.88 |
| 247 | T470V | gag | 33 | 5.6E−01 | 0.73 | 8.2E−01 | 0.88 |
| 248 | I93L | PR | 1335 | 2.8E−02 | 0.89 | 3.0E−03 | 0.88 |
| 249 | R41K | PR | 846 | 2.8E−01 | 0.91 | 4.9E−02 | 0.88 |
| 250 | H69Q | PR | 133 | 7.9E−01 | 0.92 | 5.5E−01 | 0.87 |
| 251 | I64M | PR | 63 | 8.6E−01 | 0.93 | 7.2E−01 | 0.86 |
| 252 | V82S | PR | 41 | 8.0E−03 | 6.17 | 7.4E−01 | 0.85 |
| 253 | T471S | gag | 56 | 1.0E+00 | 1.05 | 7.1E−01 | 0.85 |
| 254 | K442R | gag | 86 | 8.9E−01 | 1.12 | 5.7E−01 | 0.84 |
| 255 | I64V | PR | 613 | 1.6E−02 | 0.80 | 3.8E−02 | 0.84 |
| 256 | R464M | gag | 30 | 7.9E−01 | 0.87 | 8.1E−01 | 0.84 |
| 257 | T469M | gag | 32 | 7.9E−01 | 1.37 | 7.1E−01 | 0.83 |
| 258 | I93M | PR | 51 | 3.0E−01 | 1.70 | 7.0E−01 | 0.83 |
| 259 | P478E | gag | 55 | 5.7E−01 | 0.77 | 6.2E−01 | 0.82 |
| 260 | K442M | gag | 57 | 3.3E−01 | 0.69 | 6.3E−01 | 0.82 |
| 261 | I479E | gag | 38 | 3.9E−01 | 0.69 | 7.3E−01 | 0.82 |
| 262 | T471P | gag | 289 | 6.6E−01 | 0.92 | 1.2E−01 | 0.81 |
| 263 | H441L | gag | 118 | 2.6E−01 | 0.75 | 3.8E−01 | 0.81 |
| 264 | N37S | PR | 275 | 7.1E−02 | 0.76 | 1.2E−01 | 0.81 |
| 265 | P472del | gag | 239 | 6.2E−01 | 0.90 | 1.5E−01 | 0.81 |
| 266 | H69K | PR | 69 | 7.9E−01 | 0.90 | 5.2E−01 | 0.80 |
| 267 | I479G | gag | 50 | 8.4E−01 | 0.90 | 6.0E−01 | 0.80 |
| 268 | P453T | gag | 185 | 3.0E−01 | 0.81 | 1.7E−01 | 0.79 |
| 269 | H441P | gag | 54 | 8.5E−01 | 0.90 | 5.3E−01 | 0.79 |
| 270 | T469I | gag | 115 | 1.5E−01 | 0.69 | 2.7E−01 | 0.77 |
| 271 | L483M | gag | 314 | 1.0E−01 | 0.79 | 4.2E−02 | 0.77 |
| 272 | P478L | gag | 82 | 2.1E−01 | 0.68 | 3.8E−01 | 0.77 |
| 273 | S462I | gag | 47 | 1.2E−01 | 0.56 | 5.0E−01 | 0.77 |
| 274 | T74S | PR | 290 | 7.9E−01 | 0.94 | 2.9E−02 | 0.76 |
| 275 | E477D | gag | 83 | 8.9E−01 | 0.93 | 3.2E−01 | 0.75 |
| 276 | H441S | gag | 173 | 1.8E−01 | 0.76 | 9.1E−02 | 0.74 |
| 277 | A71T | PR | 441 | 1.6E−03 | 0.71 | 1.9E−03 | 0.74 |
| 278 | K475E | gag | 34 | 8.0E−01 | 0.88 | 5.2E−01 | 0.73 |
| 279 | H69Y | PR | 84 | 2.6E−01 | 0.71 | 2.7E−01 | 0.73 |
| 280 | G466E | gag | 32 | 5.5E−01 | 0.70 | 5.0E−01 | 0.73 |
| 281 | E477Q | gag | 32 | 6.8E−01 | 0.81 | 5.0E−01 | 0.73 |
| 282 | M36V | PR | 135 | 2.2E−03 | 0.54 | 1.1E−01 | 0.72 |
| 283 | K475R | gag | 105 | 1.4E−02 | 0.56 | 1.5E−01 | 0.72 |
| 284 | K442E | gag | 100 | 3.0E−02 | 0.59 | 1.4E−01 | 0.71 |
| 285 | S473del | gag | 41 | 1.3E−01 | 0.55 | 3.9E−01 | 0.70 |
| 286 | I479del | gag | 76 | 1.5E−01 | 0.65 | 2.0E−01 | 0.70 |
| 287 | Q476R | gag | 29 | 1.0E+00 | 0.99 | 4.8E−01 | 0.69 |
| 288 | E477K | gag | 29 | 5.3E−01 | 0.70 | 4.8E−01 | 0.69 |
| 289 | P478R | gag | 29 | 7.9E−01 | 0.83 | 4.8E−01 | 0.69 |
| 290 | S451N | gag | 466 | 7.6E−08 | 0.59 | 2.9E−05 | 0.68 |
| 291 | K70R | PR | 93 | 9.0E−03 | 0.52 | 8.4E−02 | 0.66 |
| 292 | L483V | gag | 49 | 2.9E−01 | 0.65 | 2.3E−01 | 0.65 |
| 293 | Q61D | PR | 32 | 2.6E−01 | 2.22 | 4.0E−01 | 0.65 |
| 294 | V77I | PR | 963 | 2.3E−18 | 0.60 | 3.1E−16 | 0.64 |
| 295 | K442V | gag | 43 | 1.0E−01 | 0.53 | 2.5E−01 | 0.64 |
| 296 | Q474P | gag | 80 | 1.0E+00 | 1.02 | 7.9E−02 | 0.63 |
| 297 | T12S | PR | 63 | 1.1E−01 | 0.59 | 1.3E−01 | 0.63 |
| 298 | I64L | PR | 94 | 3.4E−02 | 0.58 | 4.5E−02 | 0.62 |
| 299 | L483T | gag | 70 | 7.3E−02 | 0.57 | 9.5E−02 | 0.62 |
| 300 | E482D | gag | 196 | 2.4E−01 | 0.79 | 1.4E−03 | 0.61 |
| 301 | H441Y | gag | 200 | 2.1E−02 | 0.67 | 4.3E−04 | 0.59 |
| 302 | K20M | PR | 137 | 2.6E−01 | 0.77 | 3.0E−03 | 0.57 |
| 303 | T470I | gag | 64 | 7.8E−01 | 0.87 | 6.1E−02 | 0.57 |
| 304 | S499L | gag | 72 | 6.2E−03 | 0.47 | 3.6E−02 | 0.56 |
| 305 | Y484H | gag | 35 | 4.7E−01 | 0.69 | 1.5E−01 | 0.55 |
| 306 | H441N | gag | 89 | 5.3E−01 | 0.81 | 1.2E−02 | 0.55 |
| 307 | L63S | PR | 78 | 2.6E−02 | 0.53 | 1.8E−02 | 0.54 |
| 308 | L63A | PR | 81 | 1.1E−02 | 0.51 | 1.1E−02 | 0.53 |
| 309 | T469E | gag | 31 | 6.8E−01 | 0.77 | 1.6E−01 | 0.53 |
| 310 | T427D | gag | 43 | 1.0E−01 | 0.53 | 8.4E−02 | 0.53 |
| 311 | V75I | PR | 79 | 5.4E−02 | 0.58 | 1.4E−02 | 0.53 |
| 312 | S440P | gag | 29 | 5.3E−01 | 0.70 | 1.5E−01 | 0.52 |
| 313 | E65D | PR | 51 | 7.1E−02 | 0.53 | 4.7E−02 | 0.51 |
| 314 | L486S | gag | 108 | 8.4E−04 | 0.48 | 1.2E−03 | 0.51 |
| 315 | L63Q | PR | 60 | 1.6E−02 | 0.47 | 2.0E−02 | 0.49 |
| 316 | S498L | gag | 290 | 8.7E−07 | 0.53 | 3.8E−10 | 0.48 |
| 317 | I437L | gag | 69 | 3.2E−01 | 0.72 | 5.6E−03 | 0.47 |
| 318 | L483Q | gag | 36 | 1.0E−01 | 0.50 | 5.7E−02 | 0.47 |
| 319 | D480del | gag | 39 | 3.0E−01 | 0.63 | 4.7E−02 | 0.46 |
| 320 | P439S | gag | 39 | 9.3E−01 | 0.92 | 2.1E−02 | 0.41 |
| 321 | L483R | gag | 39 | 1.7E−01 | 0.57 | 7.8E−03 | 0.37 |
| 322 | K45R | PR | 129 | 9.8E−10 | 0.32 | 3.3E−08 | 0.35 |
| 323 | N88D | PR | 285 | 1.0E−30 | 0.26 | 2.1E−19 | 0.34 |
| 324 | K43R | PR | 38 | 5.7E−03 | 0.35 | 1.4E−03 | 0.30 |
| 325 | D30N | PR | 246 | 1.7E−48 | 0.16 | 1.0E−31 | 0.20 |
| 326 | N88S | PR | 64 | 4.1E−21 | 0.07 | 1.6E−09 | 0.17 |
| 327 | I50L | PR | 84 | 5.4E−04 | 0.42 | 1.2E−15 | 0.12 |

TABLE 2
Mutations Associated with Reduced Susceptibility to Both APV and DRV

| | | | | APV | | DRV | |
|---|---|---|---|---|---|---|---|
| | | PR or | | | Odds | | Odds |
| | Mutation | gag | n Mut | P-value | Ratio | P-value | Ratio |
| 1 | V11L | PR | 56 | 1.6E−06 | >>> | 4.3E−13 | >>> |
| 2 | I50V | PR | 104 | 2.7E−12 | >>> | 1.0E−24 | >>> |
| 3 | L89V | PR | 185 | 4.1E−22 | >>> | 1.8E−39 | 44.56 |
| 4 | A71L | PR | 47 | 1.6E−05 | >>> | 2.9E−08 | 16.53 |
| 5 | L76V | PR | 146 | 2.1E−17 | >>> | 1.9E−22 | 11.18 |
| 6 | K55N | PR | 30 | 1.4E−03 | >>> | 9.0E−05 | 10.28 |
| 7 | I54S | PR | 41 | 9.9E−05 | >>> | 2.4E−03 | 3.57 |
| 8 | I54T | PR | 41 | 9.9E−05 | >>> | 6.8E−03 | 3.03 |
| 9 | I47V | PR | 313 | 7.6E−37 | 98.74 | 6.7E−54 | 13.64 |
| 10 | V32I | PR | 396 | 3.8E−46 | 62.34 | 6.6E−77 | 18.66 |
| 11 | I84V | PR | 923 | 1.6E−124 | 58.10 | 2.5E−148 | 7.06 |
| 12 | I54M | PR | 207 | 1.1E−21 | 32.44 | 2.4E−35 | 14.47 |
| 13 | T74P | PR | 178 | 4.4E−18 | 27.85 | 4.8E−20 | 6.15 |
| 14 | V11I | PR | 148 | 1.6E−14 | 23.10 | 6.5E−24 | 12.85 |
| 15 | G73T | PR | 140 | 1.0E−13 | 21.84 | 2.6E−18 | 7.84 |
| 16 | A431I | gag | 69 | 1.1E−06 | 21.52 | 2.3E−07 | 5.60 |
| 17 | P79A | PR | 58 | 1.6E−05 | 18.04 | 3.4E−06 | 5.35 |
| 18 | L33F | PR | 900 | 7.2E−93 | 12.07 | 6.2E−140 | 6.78 |
| 19 | L24F | PR | 38 | 2.9E−03 | 11.71 | 1.7E−05 | 8.57 |
| 20 | V82F | PR | 76 | 2.2E−06 | 11.71 | 6.5E−08 | 5.47 |
| 21 | T91S | PR | 145 | 7.3E−12 | 11.16 | 1.8E−12 | 4.34 |
| 22 | F53Y | PR | 34 | 6.0E−03 | 10.44 | 7.7E−03 | 3.43 |
| 23 | E35N | PR | 99 | 7.8E−08 | 10.13 | 1.8E−05 | 2.90 |
| 24 | L449V | gag | 186 | 1.5E−14 | 9.49 | 2.2E−17 | 4.73 |
| 25 | T4A | PR | 31 | 1.3E−02 | 9.49 | 5.5E−05 | 10.65 |
| 26 | P453V | gag | 31 | 1.3E−02 | 9.49 | 5.6E−04 | 6.86 |
| 27 | P79S | PR | 31 | 1.3E−02 | 9.49 | 2.4E−03 | 4.96 |
| 28 | C67F | PR | 91 | 4.2E−07 | 9.28 | 7.4E−07 | 3.72 |
| 29 | V82C | PR | 60 | 9.9E−05 | 9.18 | 1.2E−04 | 3.67 |
| 30 | I54L | PR | 229 | 2.2E−17 | 8.74 | 4.5E−24 | 5.50 |
| 31 | T4S | PR | 53 | 5.1E−04 | 8.07 | 3.4E−03 | 2.80 |
| 32 | Q92K | PR | 179 | 8.2E−13 | 7.78 | 2.7E−09 | 2.82 |
| 33 | I54A | PR | 76 | 1.8E−05 | 7.70 | 2.8E−03 | 2.37 |
| 34 | F463S | gag | 48 | 1.7E−03 | 7.28 | 2.1E−03 | 3.18 |
| 35 | K43T | PR | 406 | 2.6E−29 | 7.24 | 2.6E−31 | 3.72 |
| 36 | L19P | PR | 43 | 3.8E−03 | 6.49 | 1.4E−05 | 7.16 |
| 37 | V82L | PR | 42 | 5.5E−03 | 6.33 | 1.1E−04 | 5.44 |
| 38 | G73A | PR | 39 | 1.2E−02 | 5.85 | 1.1E−05 | 8.81 |
| 39 | Q61N | PR | 70 | 3.9E−04 | 5.22 | 2.7E−03 | 2.48 |
| 40 | C95F | PR | 100 | 1.1E−05 | 4.96 | 4.3E−06 | 3.13 |
| 41 | F53L | PR | 314 | 1.5E−17 | 4.91 | 1.9E−11 | 2.30 |
| 42 | L23I | PR | 81 | 1.4E−04 | 4.81 | 7.7E−03 | 2.10 |
| 43 | E34Q | PR | 184 | 1.4E−09 | 4.54 | 4.5E−12 | 3.36 |
| 44 | G16A | PR | 152 | 8.9E−08 | 4.49 | 4.1E−11 | 3.73 |
| 45 | Q58E | PR | 471 | 1.6E−25 | 4.49 | 1.5E−19 | 2.41 |
| 46 | A22V | PR | 45 | 1.3E−02 | 4.43 | 6.9E−04 | 3.99 |
| 47 | K20V | PR | 90 | 1.1E−04 | 4.43 | 3.6E−06 | 3.40 |
| 48 | I66F | PR | 114 | 1.0E−05 | 4.19 | 3.5E−07 | 3.25 |
| 49 | K55R | PR | 496 | 2.6E−25 | 4.17 | 2.7E−25 | 2.70 |
| 50 | I66V | PR | 99 | 5.8E−05 | 4.16 | 2.1E−09 | 4.86 |
| 51 | P453I | gag | 56 | 4.9E−03 | 4.11 | 1.8E−06 | 6.12 |
| 52 | T4P | PR | 41 | 2.4E−02 | 4.01 | 6.8E−03 | 3.03 |
| 53 | H69R | PR | 95 | 1.2E−04 | 3.98 | 1.4E−04 | 2.59 |
| 54 | I72L | PR | 133 | 1.2E−05 | 3.51 | 1.0E−07 | 3.02 |
| 55 | I437V | gag | 435 | 1.3E−18 | 3.51 | 2.4E−11 | 1.95 |
| 56 | L10F | PR | 615 | 3.4E−26 | 3.36 | 1.2E−30 | 2.59 |
| 57 | I85V | PR | 290 | 6.6E−11 | 3.21 | 1.3E−14 | 2.76 |
| 58 | A431V | gag | 1181 | 2.2E−59 | 3.21 | 1.0E−40 | 1.94 |
| 59 | V82T | PR | 224 | 5.7E−08 | 3.06 | 5.3E−05 | 1.84 |
| 60 | H441Q | gag | 245 | 1.3E−08 | 3.05 | 1.3E−06 | 1.99 |
| 61 | G73C | PR | 73 | 6.7E−03 | 2.98 | 5.1E−03 | 2.24 |
| 62 | E468K | gag | 73 | 6.7E−03 | 2.98 | 5.1E−03 | 2.24 |
| 63 | R452S | gag | 171 | 1.1E−05 | 2.87 | 2.9E−13 | 3.92 |
| 64 | L89I | PR | 67 | 2.3E−02 | 2.71 | 1.1E−02 | 2.16 |
| 65 | G73S | PR | 389 | 7.2E−12 | 2.69 | 1.8E−09 | 1.91 |
| 66 | I54V | PR | 1189 | 3.3E−47 | 2.67 | 6.6E−13 | 1.42 |
| 67 | P459T | gag | 99 | 5.7E−03 | 2.53 | 3.1E−02 | 1.69 |
| 68 | R452K | gag | 71 | 2.8E−02 | 2.49 | 3.0E−04 | 2.99 |
| 69 | M46L | PR | 512 | 4.6E−14 | 2.48 | 1.9E−07 | 1.60 |
| 70 | K20R | PR | 731 | 1.6E−20 | 2.41 | 2.2E−19 | 1.90 |
| 71 | E467K | gag | 77 | 2.5E−02 | 2.39 | 2.1E−03 | 2.41 |
| 72 | T12P | PR | 119 | 3.4E−03 | 2.37 | 5.1E−03 | 1.84 |
| 73 | M46I | PR | 1283 | 2.1E−42 | 2.32 | 1.9E−45 | 1.92 |
| 74 | V82I | PR | 116 | 5.8E−03 | 2.31 | 1.9E−04 | 2.31 |
| 75 | F463V | gag | 74 | 3.2E−02 | 2.29 | 2.7E−04 | 2.89 |
| 76 | S462N | gag | 103 | 1.3E−02 | 2.19 | 5.6E−04 | 2.29 |
| 77 | F465S | gag | 165 | 1.3E−03 | 2.17 | 8.6E−07 | 2.45 |
| 78 | M423I | gag | 136 | 6.2E−03 | 2.07 | 4.5E−04 | 2.04 |
| 79 | M36L | PR | 196 | 1.4E−03 | 1.98 | 1.1E−06 | 2.20 |
| 80 | V82A | PR | 1043 | 3.9E−22 | 1.98 | 4.6E−02 | 1.12 |
| 81 | L89M | PR | 165 | 4.7E−03 | 1.95 | 2.8E−02 | 1.51 |
| 82 | L10I | PR | 1382 | 1.9E−33 | 1.95 | 1.5E−09 | 1.29 |
| 83 | E428D | gag | 107 | 2.9E−02 | 1.94 | 1.5E−04 | 2.41 |
| 84 | A71I | PR | 256 | 2.0E−04 | 1.93 | 6.8E−05 | 1.74 |
| 85 | S451T | gag | 123 | 3.2E−02 | 1.85 | 7.7E−03 | 1.78 |
| 86 | S488A | gag | 206 | 4.5E−03 | 1.79 | 8.8E−06 | 2.02 |
| 87 | F465L | gag | 186 | 2.1E−02 | 1.65 | 2.9E−03 | 1.66 |
| 88 | A71V | PR | 1411 | 1.4E−20 | 1.62 | 6.9E−21 | 1.47 |
| 89 | L10V | PR | 441 | 1.2E−04 | 1.62 | 3.0E−03 | 1.36 |
| 90 | I15V | PR | 751 | 7.0E−08 | 1.60 | 1.1E−09 | 1.53 |
| 91 | D425E | gag | 451 | 1.9E−04 | 1.59 | 5.6E−05 | 1.49 |
| 92 | P453L | gag | 919 | 7.6E−10 | 1.58 | 1.1E−22 | 1.80 |
| 93 | F463L | gag | 316 | 4.6E−03 | 1.57 | 2.0E−05 | 1.68 |
| 94 | K20T | PR | 254 | 1.6E−02 | 1.55 | 2.3E−03 | 1.54 |
| 95 | N37D | PR | 636 | 9.2E−06 | 1.55 | 6.9E−04 | 1.31 |
| 96 | D60E | PR | 485 | 4.9E−04 | 1.51 | 5.8E−04 | 1.39 |
| 97 | I72V | PR | 433 | 1.2E−03 | 1.51 | 1.6E−02 | 1.29 |
| 98 | M36I | PR | 1447 | 5.2E−15 | 1.48 | 8.0E−15 | 1.37 |
| 99 | L449F | gag | 269 | 3.2E−02 | 1.46 | 7.8E−03 | 1.44 |
| 100 | I13V | PR | 1282 | 7.7E−10 | 1.41 | 1.2E−31 | 1.71 |
| 101 | I479T | gag | 516 | 3.1E−02 | 1.28 | 4.2E−03 | 1.30 |
| 102 | I62V | PR | 1600 | 4.3E−08 | 1.28 | 1.4E−05 | 1.17 |
| 103 | E35D | PR | 1165 | 9.3E−05 | 1.27 | 2.7E−08 | 1.32 |
| 104 | L90M | PR | 1681 | 1.8E−07 | 1.24 | 4.9E−11 | 1.25 |
| 105 | L63P | PR | 2392 | 1.5E−05 | 1.10 | 2.2E−04 | 1.07 |

TABLE 3
Mutations Associated with Increased Susceptibility to Both APV and DRV

| | | | | APV | | DRV | |
|---|---|---|---|---|---|---|---|
| | | PR or | | | Odds | | Odds |
| | Mutation | gag | n Mut | P-value | Ratio | P-value | Ratio |
| 1 | N88S | PR | 64 | 4.1E−21 | 0.07 | 1.57E−09 | 0.17 |
| 2 | D30N | PR | 246 | 1.7E−48 | 0.16 | 1.02E−31 | 0.20 |
| 3 | N88D | PR | 285 | 1.0E−30 | 0.26 | 2.12E−19 | 0.34 |
| 4 | K45R | PR | 129 | 9.8E−10 | 0.32 | 3.33E−08 | 0.35 |
| 5 | K43R | PR | 38 | 5.7E−03 | 0.35 | 1.42E−03 | 0.30 |
| 6 | I50L | PR | 84 | 5.4E−04 | 0.42 | 1.16E−15 | 0.12 |
| 7 | S499L | gag | 72 | 6.2E−03 | 0.47 | 3.59E−02 | 0.56 |
| 8 | L63Q | PR | 60 | 1.6E−02 | 0.47 | 1.96E−02 | 0.49 |
| 9 | L486S | gag | 108 | 8.4E−04 | 0.48 | 1.19E−03 | 0.51 |
| 10 | L63A | PR | 81 | 1.1E−02 | 0.51 | 1.14E−02 | 0.53 |
| 11 | S498L | gag | 290 | 8.7E−07 | 0.53 | 3.79E−10 | 0.48 |
| 12 | L63S | PR | 78 | 2.6E−02 | 0.53 | 1.83E−02 | 0.54 |
| 13 | I64L | PR | 94 | 3.4E−02 | 0.58 | 4.48E−02 | 0.62 |
| 14 | S451N | gag | 466 | 7.6E−08 | 0.59 | 2.94E−05 | 0.68 |
| 15 | V77I | PR | 963 | 2.3E−18 | 0.60 | 3.08E−16 | 0.64 |
| 16 | H441Y | gag | 200 | 2.1E−02 | 0.67 | 4.30E−04 | 0.59 |
| 17 | A71T | PR | 441 | 1.6E−03 | 0.71 | 1.88E−03 | 0.74 |
| 18 | I64V | PR | 613 | 1.6E−02 | 0.80 | 3.85E−02 | 0.84 |
| 19 | I93L | PR | 1335 | 2.8E−02 | 0.89 | 3.03E−03 | 0.88 |

1. A method for determining whether a human immunodeficiency virus type 1 (HIV-1) is likely to have a reduced susceptibility to darunavir, comprising: determining the susceptibility of the HIV-1 to amprenavir, wherein reduced susceptibility to amprenavir correlates with reduced susceptibility to darunavir, thereby determining whether the HIV-1 is likely to have a reduced susceptibility to darunavir.

2. (canceled)

3. The method of claim 1, wherein the susceptibility to amprenavir is determined by measuring in vitro the sensitivity of the HIV-1 to amprenavir.

4. The method of claim 1, wherein the susceptibility to amprenavir is determined by detecting, in a gene encoded by the HIV-1, the presence of one or more mutations associated with reduced susceptibility to amprenavir.

5. The method of claim 4, wherein said one or more mutations is in at least one of codons 4, 10, 11, 12, 13, 15, 16, 19, 20, 22, 23, 24, 32, 34, 35, 36, 37, 43, 46, 47, 50, 53, 54, 55, 58, 60, 61, 62, 63, 66, 67, 69, 71, 72, 73, 74, 76, 79, 82, 84, 85, 89, 90, 91, 92 and 95 of the protease gene of the HIV-1.

6. The method of claim 4, wherein said one or more mutations are selected from the group consisting of T4A, T4P, T4S, L10F, L10V, L10I, V11L, V11I, T12P, I13V, I15V, G16A, L19P, K20V, K20R, K20T, A22V, L23I, L24F, V32I, L33F, E34Q, E35N, E35D, M36L, M36I, N37D, K43T, M46I, M46L, I47V, I50V, F53Y, F53L, 154M, I54L, I54S, I54T, I54A, I54V, K55N, K55R, Q58E, D60E, Q61N, I62V, L63P, I66V, I66F, C67F, H69R, A71L, A71I, A71V, I72L, I72V, G73A, G73T, G73C, G73S, T74P, L76V, P79A, P79S, V82F, V82L, V82C, V82I, V82T, V82A, 184V, 185V, L89V, L89I, L89M, L90M, T91S, Q92K and C95F.

7. The method of claim 4, wherein said one or more mutations is in at least one of codons 423, 425, 428, 431, 437, 441, 449, 451, 452, 453, 459, 462, 463, 465, 467, 468, 479 and 488 of the gag gene of the HIV-1.

8. The method of claim 4, wherein said one or more mutations is selected from the group consisting of M423I, D425E, E428D, A431I, A431V, I437V, H441Q, L449V, L449F, S451T, R452S, R452K, P453V, P453I, P453L, P459T, S462N, F463S, F463V, F463L, F465S, F465L, E467K, E468K, I479T and S488A.

9. The method of claim 1, wherein the HIV-1 is an HIV-1 isolated from a patient sample.

10. The method of claim 1, wherein the HIV-1 is isolated from the patient sample without passage through cell culture.

11. A method for determining whether a human immunodeficiency virus type 1 (HIV-1) is likely to have an increased susceptibility to darunavir, comprising: determining the susceptibility of the HIV-1 to amprenavir, wherein increased susceptibility to amprenavir correlates with increased susceptibility to darunavir, thereby determining whether the HIV-1 is likely to have an increased susceptibility to darunavir.

12. (canceled)

13. The method of claim 11, wherein the susceptibility to amprenavir is determined by measuring in vitro the sensitivity of the HIV-1 to amprenavir.

14. The method of claim 11, wherein the susceptibility to amprenavir is determined by detecting, in a gene encoded by the HIV-1, the presence of one or more mutations associated with increased susceptibility to amprenavir.

15. The method of claim 13, wherein said one or more mutations is in at least one of codons 30, 43, 45, 50, 63, 64, 71, 77, 88 and 93 of the protease gene of the HIV-1.

16. The method of claim 13, wherein said one or more mutations are selected from the group consisting of D30N, K43R, K45R, I50L, L63S, L63A, L63Q, 164V, I64L, A71T, V77I, N88D, N88S and I93L.

17. The method of claim 13, wherein said one or more mutations is in at least one of codons 441, 451, 486, 498 and 499 of the gag gene of the HIV-1.

18. The method of claim 13, wherein said one or more mutations is selected from the group consisting of H441Y, S451N, L486S, S498L and S499L.

19. The method of claim 11, wherein the HIV-1 is an HIV-1 isolated from a patient sample.

20. The method of claim 11, wherein the HIV-1 is isolated from the patient sample without passage through cell culture.

21. A method for determining whether an HIV-1 is likely to have a reduced susceptibility to darunavir, comprising: detecting whether an HIV-1 protease mutation is present in at least one of codons 4, 10, 12, 13, 15, 16, 18, 19, 20, 22, 23, 24, 34, 35, 36, 37, 43, 46, 48, 53, 55, 57, 58, 60, 61, 62, 63, 66, 67, 69, 71, 72, 74, 79, 82, 83, 85, 90, 91, 92, and 95 of the HIV-1, wherein the presence of the mutation(s) correlates with reduced susceptibility to treatment with darunavir, thereby determining whether the HIV-1 is likely to have a reduced susceptibility to darunavir.

22. (canceled)

23. The method of claim 21, wherein the mutation at codon 4 encodes alanine (A) or proline (P), the mutation at codon 16 encodes alanine (A), the mutation at codon 19 encodes proline (P), the mutation at codon 20 encodes valine (V), the mutation at codon 22 encodes valine (V), the mutation at codon 24 encodes phenylalanine (F), the mutation at codon 34 encodes glutamine (Q), the mutation at codon 43 encodes threonine (T), the mutation at codon 53 encodes tyrosine (Y), the mutation at codon 55 encodes asparagine (N), the mutation at codon 66 encodes valine (V) or phenylalanine (F), the mutation at codon 67 encodes phenylalanine (F), the mutation at codon 71 encodes leucine (L), the mutation at codon 72 encodes leucine (L), the mutation at codon 74 encodes proline (P), the mutation at codon 79 encodes alanine (A) or serine (S), the mutation at codon 82 encodes phenylalanine (F), leucine (L) or cysteine (C), the mutation at codon 91 encodes serine (S), or the mutation at codon 95 encodes phenylalanine (F).

24-41. (canceled)

42. The method of claim 21, further comprising detecting whether said HIV-1 protease mutation(s) are present in combination with a mutation in at least one of codons 11, 32, 33, 47, 50, 54, 73, 76, 84 and 89.

43. The method of claim 42, wherein the mutation at codon 11 encodes isoleucine (I), the mutation at codon 32 encodes isoleucine (I), the mutation at codon 33 encodes phenylalanine (F), the mutation at codon 47 encodes valine (V), the mutation at codon 50 encodes valine (V), the mutation at codon 54 encodes leucine (L), methionine (M), serine (S) or threonine (T), the mutation at codon 73 encodes alanine (A), serine (S) or threonine (T), the mutation at codon 76 encodes valine (V), the mutation at codon 84 encodes valine (V), or the mutation at codon 89 encodes valine (V).

44-52. (canceled)

53. A method for determining whether an HIV-1 is likely to have a reduced susceptibility to darunavir, comprising: detecting whether an HIV-1 gag mutation is present in at least one of codons 423, 425, 428, 431, 435, 437, 441, 449, 451, 452, 453, 459, 462, 463, 465, 467, 468, 469, 479, 488 and 497 of the HIV-1, wherein the presence of the mutation(s) correlates with reduced susceptibility to treatment with darunavir, thereby determining whether the HIV-1 is likely to have a reduced susceptibility to darunavir.

54. (canceled)

55. The method of claim 53, wherein the mutation at codon 431 encodes isoleucine (I), the mutation at codon 449 encodes valine (V), the mutation at codon 452 encodes serine (S), the mutation at codon 453 encodes valine (V) or isoleucine (I), or the mutation at codon 463 glutamic acid (E) or serine (S).

56-59. (canceled)

60. A method for determining whether an HIV-1 is likely to have an increased susceptibility to darunavir, comprising: detecting whether an HIV-1 protease mutation is present in at least one of codons 20, 30, 36, 41, 43, 45, 50, 63, 64, 65, 70, 71, 74, 75, 77, 82, 88 and 93 of the HIV-1, wherein the presence of the mutation(s) correlates with increased susceptibility to treatment with darunavir, thereby determining whether the HIV-1 is likely to have an increased susceptibility to darunavir.

61. (canceled)

62. The method of claim 60, wherein the mutation at codon 43 is arginine (R), the mutation at codon 45 is arginine (R), or the mutation at codon 63 is glutamine (Q).

63-64. (canceled)

65. The method of claim 60, further comprising detecting whether said HIV-1 protease mutation(s) are present in combination with a mutation in at least one of codons 30, 50 or 88.

66. The method of claim 65, wherein the mutation at codon 30 encodes asparagine (N), the mutation at codon 50 encodes leucine (L), or the mutation at codon 88 encodes aspartic acid (D) or serine (S).

67-68. (canceled)

69. A method for determining whether an HIV-1 is likely to have an increased susceptibility to darunavir, comprising: detecting whether an HIV-1 gag mutation is present in at least one of codons 437, 439, 441, 442, 451, 475, 480, 482, 483, 486, 498 and 499 of the HIV-1, wherein the presence of the mutation(s) correlates with increased susceptibility to treatment with darunavir, thereby determining whether the HIV-1 is likely to have an increased susceptibility to darunavir.

70. (canceled)

71. The method of claim 69, wherein the mutation at codon 437 encodes leucine (L), the mutation at codon 439 encodes serine (S), the mutation at codon 480 results in deletion of the codon, the mutation at codon 483 encodes arginine (R), or the mutation at codon 498 encodes leucine (L).

72-75. (canceled)
